(19) United States
(12) Patent Application Publication
(10) Pub. No.: US 2024/0189147 A1
(43) Pub. Date: Jun. 13, 2024

(54) GLAUCOMA TREATMENT SYSTEMS AND PROCEDURES

(71) Applicant: AqueSys, Inc., North Chicago, IL (US)

(72) Inventor: Laszlo Romoda, Mission Viejo, CA (US)

(21) Appl. No.: 18/568,157

(22) PCT Filed: Jun. 30, 2022
(86) PCT No.: PCT/US2022/035877

§ 371 Date: Dec. 7, 2023

Related U.S. Application Data
(60) Provisional application No. 63/217,230, filed on Jun. 30, 2021.

(51) Int. Cl. A61F 9/007

(52) U.S. Cl. CPC A61F 9/00781

(57) ABSTRACT

Glaucoma treatment can be performed using an intraocular shunt inserter to deliver an intraocular shunt. The shunt can have at least one flow path having an inner diameter of about 50 μm and about 70 μm and between about 170 μm to about 260 μm. The inserter can carry the intraocular shunt within a 27 Gauge needle having a lumen with an inner diameter of between about 220 to about 280 μm. Embodiments of these systems and related procedures can safely and significantly lower glaucoma patient's intraocular pressure and reduce the number of antiglaucoma medications.

### CROSS-REFERENCE TO RELATED APPLICATIONS

The present application claims priority to and the benefit of U.S. Provisional App. No. 63/217,230, filed on Jun. 30, 2021, the entirety of which is incorporated herein by reference.

### BACKGROUND

### Field of the Inventions

The present inventions relate to intraocular shunts, intraocular inserters and their use in methods of implanting an intraocular shunt into an eye.

### Description of the Related Art

Glaucoma is a disease of the eye that affects millions of people. Glaucoma refers to a wide range of multifactorial, chronic, and progressive optic neuropathies, characterized by progressive loss of retinal ganglion cells and subsequent visual field defects. Glaucoma is associated with an increase in intraocular pressure resulting either from a failure of a drainage system of an eye to adequately remove aqueous humor from an anterior chamber of the eye or overproduction of aqueous humor by a ciliary body in the eye. Build-up of aqueous humor and resulting intraocular pressure may result in irreversible damage to the optic nerve and the retina, which may lead to irreversible retinal damage and blindness.

Glaucoma may be treated in a number of different ways. One manner of treatment involves delivery of drugs such as beta-blockers or prostaglandins to the eye to either reduce production of aqueous humor or increase flow of aqueous humor from an anterior chamber of the eye. Glaucoma filtration surgery is a surgical procedure typically used to treat glaucoma. The procedure involves placing a shunt in the eye to relieve intraocular pressure by creating a pathway for draining aqueous humor from the anterior chamber of the eye. The shunt is typically positioned in the eye such that it creates a drainage pathway between the anterior chamber of the eye and a region of lower pressure. Such fluid flow pathways allow for aqueous humor to exit the anterior chamber.

### SUMMARY

Glaucoma treatment can be performed using an intraocular shunt inserter to deliver an intraocular shunt. The shunt can have at least one flow path having an inner diameter of about 50 μm and about 70 μm and between about 170 μm to about 260 μm. The inserter can carry or hold the intraocular shunt within a 27 Gauge needle having a lumen with an inner diameter of between about 220 to about 280 μm. Embodiments of these systems and related procedures can safely and significantly lower glaucoma patient's intraocular pressure and reduce the number of antiglaucoma medications.

In accordance with some embodiments, a system for treating glaucoma is provided that can comprise an intraocular shunt and a needle. The intraocular shunt can be made of a cross-linked gelatin. The intraocular shunt can have a shunt outer diameter of between about 170 μm to about 260 μm. The intraocular shunt can define at least one interior flow path having a shunt inner diameter of between about 50 μm and about 70 μm. The needle can have a lumen to carry or hold the intraocular shunt. The lumen can have a lumen inner diameter of between about 220 to about 280 μm.

In some embodiments, the system can have an intraocular shunt that defines a shunt inner diameter of about 63 μm. Further, the needle can have a needle outer diameter of between about 400 μm to about 420 μm, and the lumen can have a lumen inner diameter of about 260 μm.

The system can be configured such that an annular gap exists between an outer surface of the shunt and an inner surface of the lumen is between about 0 μm to about 20 μm. Further, the shunt outer diameter can be expandable. For example, the shunt outer diameter can expand to a maximum shunt outer diameter of about 260 μm.

In some embodiments, the shunt inner diameter can be about 63 μm. Optionally, the intraocular shunt can have a shunt wall thickness of between about 88 μm to about 99 μm. Further, the intraocular shunt can have a shunt length of about 6 millimeters.

In some embodiments, the needle's lumen inner diameter can be about 260 μm. The needle can also have a needle outer diameter of between about 400 μm to about 420 μm. The needle can have a needle wall thickness of between about 70 μm to about 80 μm.

Optionally, the system can further comprise a movable plunger disposed within the lumen. The plunger can be configured to advance the shunt through the lumen.

In some embodiments, the system can further comprise a deployment mechanism that is configured to selectively move the plunger to advance the shunt.

Methods of treating glaucoma also provided. For example, a method can be performed that comprises: advancing a needle through the sclera of the patient to create an opening, wherein the needle has a needle diameter of between about 400 μm to about 420 μm; advancing an intraocular shunt through a lumen of the needle and into the opening, wherein the intraocular shunt has a shunt outer diameter of between about 240 to 260 μm and the lumen has a lumen inner diameter of between about 220 μm to about 280 μm for reducing any gap between the shunt and the opening to minimize peritubular flow therebetween; and permitting flow through the intraocular shunt, wherein the shunt has a shunt inner diameter of between about 50 μm to about 70 μm.

The method can also comprise permitting expansion of the intraocular shunt. For example, the shunt outer diameter can be limited to approximately the lumen inner diameter during expansion. Further, the shunt outer diameter can be expanded to approximately an inner diameter of the opening when the shunt is outside of the lumen of the needle.

The method may optionally involve cutting the sclera via a tip of the needle to form the opening.

### BRIEF DESCRIPTION OF THE DRAWINGS

The accompanying drawings, which are included to provide further understanding of the subject technology and are incorporated in and constitute a part of this specification, illustrate aspects of the disclosure and together with the description serve to explain the principles of the subject technology.

FIG. 1A is a schematic view of a procedure for implanting an intraocular shunt into an eye using an inserter, according to some embodiments.

FIG. 1B is a side, cross-sectional view of an ab interno procedure for implanting an intraocular shunt into an eye using an inserter, according to some embodiments.

FIG. 1C is a side, cross-sectional view of an ab externo procedure for implanting an intraocular shunt into an eye using an inserter, according to some embodiments.

FIG. 2 is a perspective view of an inserter for implanting an intraocular shunt into an eye, according to some embodiments.

FIG. 3 is a perspective, exploded view of the inserter shown in FIG. 2, according to some embodiments.

FIG. 4 is a perspective, exploded view of a drive assembly of the inserter shown in FIG. 2, according to some embodiments.

FIG. 5 is a scatter plot of the intraocular pressure of patients in a study, who underwent a procedure pursuant to some embodiments of the present disclosure, at baseline and month 3.

FIG. 6 is a scatter plot of the intraocular pressure of patients in a study, who underwent a procedure pursuant to some embodiments of the present disclosure, at baseline and through month 18.

FIG. 7 is an overview of the mean intraocular pressure and number of hypotensive medications used by the patients, who underwent a procedure pursuant to some embodiments of the present disclosure, at month 3 of the study.

FIG. 8 is an overview of the mean intraocular pressure and number of hypotensive medications used by the patients, who underwent a procedure pursuant to some embodiments of the present disclosure, over the course of the study.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Disclosed herein is the unique combination of a modified intraocular implant with a modified insertion device, referred to collectively herein as the “XEN63 system.”

Embodiments of this unique XEN63 system have demonstrated the potential for achieving unexpectedly improved, more effective, glaucoma treatment, with reduced adverse events in comparison to prior systems. The XEN® implant or shunt is a commercially available intraocular shunt that can be implanted into the human eye for treating glaucoma. The XEN® has a 45 μm inner diameter (hereinafter “XEN45 shunt”). However, as explained further herein, Applicant's research and development team, in an effort to improve an already successful procedure, discovered surprising changes in the efficacy of glaucoma treatment using a specific combination of shunts and inserters that are different from the prior combination of shunts and inserters in various respects. Specifically, the XEN63 system comprises a modified version of the XEN45 shunt, which can have, e.g., a 63 μm inner diameter (the 63 μm inner diameter shunt is hereinafter referred to as the “XEN63 shunt”). Further, the XEN63 system uses an inserter that was originally designed for use only with the XEN45 shunt, but which had been modified in a number of respects, which include, e.g., a specially modified, 27 Gauge needle. As explained herein, the XEN63 system provides surprising, unexpected benefits that are superior to prior systems or devices.

For example, the collective effect of delivering the XEN63 shunt using an inserter with a modified 27 Gauge needle provides: an immediate, clear, and substantial reduction in intraocular pressure (“IOP”), particularly in patients with higher baseline IOP values, without a correspondingly increased risk of developing hypotony compared to the XEN45 shunt despite having a significantly larger inner lumen diameter; a low risk for postoperative needling compared to the XEN45 shunt; a reduced use of hypotensive antiglaucoma medications post insertion compared to the XEN45 shunt; and comparable incidence of peritubular filtration to the XEN45 inserter. Overall, the XEN63 system significantly lowered IOP and reduced the number of antiglaucoma medications, with a good safety profile. The XEN63 system's limited incidence of hypotony, in combination with the optional use of MMC (both in terms of concentration and area of injection), may allow to treat more advanced patients and to obtain a lower target-IOP in the long-term.

### Glaucoma Treatment Procedures and Systems

Glaucoma is a disease in which the optic nerve is damaged, leading to progressive, irreversible loss of vision. It is typically associated with increased pressure of the fluid (i.e., aqueous humor) in the eye. Untreated glaucoma leads to permanent damage of the optic nerve and resultant visual field loss, which can progress to blindness. Once lost, this damaged visual field cannot be recovered. The main goal of glaucoma treatment is to slow the progression of the disease and preserve, as much as possible, the patient's quality of life, immediate and consistent reduction in IOP becomes a paramount consideration in assessing the effectiveness and viability of a surgical option for treating glaucoma.

When drug therapy fails, or is not tolerated, surgical intervention is warranted. There are various surgical filtration methods for lowering IOP by creating a fluid flow-path between the anterior chamber and the subconjunctival tissue. In one particular method, an intraocular shunt is implanted with an inserter by directing a needle, which carries or holds the shunt through the cornea, across the anterior chamber, and through the trabecular meshwork and sclera, and into the subconjunctival space. See, for example, U.S. Pat. No. 6,544,249, U.S. Patent Pub. No. 2008/0108933, and U.S. Pat. No. 6,007,511, the entireties of each of which are incorporated herein by reference.

Surgeons may implant the intraocular shunt using an ab externo procedure or an ab interno procedure, depending on the needs of the patient and the selected treatment. In an ab externo approach, the surgeon may enter through the conjunctiva and inwards through the sclera, as described in the present applicant's publications, including U.S. Pat. Nos. 10,470,927 and 10,463,537, the entireties of each of which are incorporated herein by reference. In an ab interno approach, the surgeon may enter through the cornea, across the anterior chamber, through the trabecular meshwork and sclera, as shown in Yu et al. (U.S. Pat. No. 6,544,249 and U.S. Patent Pub. No. 2008/0108933) and Prywes (U.S. Pat. No. 6,007,511), the contents of each of which are incorporated by reference herein in its entirety.

In order to assess the efficacy and safety of intraocular shunts, inserters, and various procedures, Applicant has conducted various studies and evaluations. In accordance with an aspect of at least some of the embodiments disclosed herein is the realization that various unexpected results and unexpected benefits were achieved using based on specific shunt and inserter characteristics. As discussed further herein, pre-study evaluations revealed skepticism and low expectations with regard to certain characteristics, which yielded surprisingly and consistently superior results, which completely swayed surgeon opinion in their favor. Accordingly, while a certain procedure and device was preferred prior to Applicant's evaluations and studies discussed herein, an overwhelming majority of surgeons has demonstrated almost uniform adoptability of embodiments of the XEN63 system disclosed herein, the newly discovered benefits and advantages to patients being exceedingly persuasive to the subject group of surgeons.

Up to now, decreasing IOP has been the only proven method to treat glaucoma. To do so, different treatment strategies, such as medical therapy, laser, and surgery are currently available. Although topical hypotensive medication is usually the first treatment approach, many patients do not achieve an adequate glaucoma control due to different causes, including poor adherence, side effects, or lack of maintained efficacy. Despite trabeculectomy is considered to be the gold standard in glaucoma surgery, due mainly to its well-established efficacy in terms of lowering IOP, it may lead to potential vision-threatening complications. Minimally invasive glaucoma surgery (MIGS) devices have been developed as a safer and less traumatic mean of lowering IOP in patients with glaucoma.

Among the different MIGS devices, the already commercialized XEN45 shunt allows flow of aqueous humor from the anterior chamber to the subconjunctival space. The XEN45 shunt has been used for years, providing effective treatment for patients suffering from glaucoma. The XEN45 shunt, which many surgeons credited with providing strong protection against hypotony, which can cause various deleterious effects, including corneal decompensation, accelerated cataract formation, maculopathy, discomfort, and overall decreased vision. Hypotony can occur when aqueous humor is permitted to drain too quickly from the anterior chamber via the intraocular shunt, causing the IOP to drop to 5 mm Hg or less.

An aspect of at least some of the embodiments disclosed herein is the realization that modifying the XEN45 shunt to have a larger diameter lumen, along with the inserter through which the XEN45 shunt is delivered, had the potential for achieving better, more effective, glaucoma treatment. However, the response or behavior of the human eye is not well understood by surgeons, and therefore, modifications to any procedures, implants, or inserters do not provide predictable results or meet the expectations of all surgeons. Moreover, the XEN45 shunt has a substantial degree of trust in the industry, given the benefits and efficacy that it provides. Therefore. Applicant's development and pathway to discovery in this clinical space has required not only extensive time and trial, but also the consideration and implementation of contrarian thought and unproven concepts, as discussed further herein.

Accordingly, some embodiments provide an intraocular shunt and delivery system that improves the reduction of IOP, reduces the reliance on ocular hypotensive drugs, and minimizes postoperative needling to maintain the reduced IOP. Such benefits and advantages can be achieved by delivering a shunt having a 63 μm inner diameter using a 27 Gauge needle.

Optionally, the shunt outer diameter can have a reduced tolerance compared to conventional shunt manufacturing procedures. The shunt can have an outer diameter of about 160 μm to about 200 μm, about 170 μm, about 180 μm, or about 190 μm. In some embodiments, the shunt can define an outer diameter of about 170 μm to about 180 μm, and preferably, about 170 μm.

These and other embodiments have been tested and studied, proving effective in terms of reducing and maintaining IOP and improving postoperative prognoses and care, as well as the reduction in adverse postoperative events. The present disclosure contemplates these challenges, provides solutions to these challenges, and provides a unique surgical system that can improve surgical outcomes for both the surgeon and patient.

Some embodiments of the methods disclosed herein can utilize an inserter or deployment device to deploy the intraocular shunt. A hollow shaft or needle can be coupled to the inserter or be part of the inserter itself. The inserter can include devices such as those as described in co-owned U.S. Pat. Nos. 9,585,790, 8,721,792, 8,852,136, and U.S. Patent Application Publication No. 2012/0123434, filed on Nov. 15, 2010, the contents of each of which are incorporated by reference herein in their entireties.

In accordance with some embodiments, the inserter can be advanced into the eye via an ab-interno or an ab-externo approach. Thereafter, the shunt can be deployed from the shaft into the eye such that the shunt forms a passage from the anterior chamber into an area of lower pressure, such as Schlemm's canal, the subconjunctival space, the episcleral vein, the suprachoroidal space, the intra-Tenon's adhesion space, the subarachnoid space, or other areas of the eye. The hollow shaft is then withdrawn from the eye. Methods for delivering and implanting bioabsorbable or permanent tubes or shunts, as well as implantation devices for performing such methods, are generally disclosed in applicant's applications, including U.S. Patent Application Publication Nos. 2012/0197175, 2015/0011926, and 2016/0354244. U.S. patent application Ser. No. 15/613,018, as well as in U.S. Pat. Nos. 6,007,511, 6,544,249, 8,852,136, and 9,585,790 each of which are incorporated by reference in their entireties.

Some methods can be conducted by making an incision in the eye prior to insertion of the inserter. However, in some instances, the method may be conducted without making an incision in the eye prior to insertion of the inserter. In some embodiments, the shaft that is connected to the inserter has a sharpened point or tip. In some embodiments, the hollow shaft is a needle. Exemplary needles that may be used are commercially available from Terumo Medical Corp. (Elkington, Md). In some embodiments, the needle can have a hollow interior and a beveled tip, and the intraocular shunt can be held within the hollow interior of the needle. In some embodiments, the needle can have a hollow interior and a triple ground point or tip.

Some methods can be conducted without needing to remove an anatomical portion or feature of the eye, including but not limited to the trabecular meshwork, the iris, the cornea, or aqueous humor. Some methods can be conducted without inducing substantial ocular inflammation, such as subconjunctival blebbing or endophthalmitis. Some methods can be achieved using an ab interno approach by inserting the hollow shaft configured to carry or hold the intraocular shunt as it is advanced through the cornea, across the anterior chamber, through the trabecular meshwork, and into the intra-scleral or intra-Tenon's adhesion space. However, some methods may be conducted using an ab externo approach.

In some methods conducted using an ab interno approach, the angle of entry through the cornea can be altered to affect optimal placement of the shunt in the intra-Tenon's adhesion space. The hollow shaft can be inserted into the eye at an angle above or below the corneal limbus, in contrast with entering through the corneal limbus.

For example, the hollow shaft can be inserted from about 0.25 mm to about 3.0 mm above the corneal limbus. The shaft can be inserted from about 0.5 mm to about 2.5 mm above the corneal limbus. The shaft can also be inserted from about 1.0 mm to about 2.0 mm above the corneal limbus, or any specific value within any of these ranges. For example, the hollow shaft can be inserted above the corneal limbus at distances of about: 1.0 mm, 1.1 mm, 1.2 mm, 1.3 mm, 1.4 mm, 1.5 mm, 1.6 mm, 1.7 mm, 1.8 mm, 1.9 mm, or 2.0 mm.

An ab interno approach or procedure may require or benefit from a shunt that is positioned differently relative to the bevel of the needle compared to the position of the shunt for ab externo procedures. For example, for an ab externo procedure it may be beneficial if the distal end of the shunt is positioned further proximal to the bevel of the needle than for an ab interno procedure. As noted above, existing inserters may not be able to, or may not easily allow for a surgeon to reposition the shunt relative to the bevel of the needle prepare for an ab externo and/or ab interno procedure. The present disclosure provides various embodiments of methods and devices that can enable an operator easily position the shunt relative to the bevel of the needle to configure the inserter for either an ab externo procedure or an ab interno procedure.

Further, in some embodiments, placement of the shunt farther from the limbus at the exit site, as provided by an angle of entry above the limbus, can provide access to more lymphatic channels for drainage of aqueous humor, such as the episcleral lymphatic network, in addition to the conjunctival lymphatic system. A higher angle of entry also results in flatter placement in the intra-Tenon's adhesion space so that there is less bending of the shunt.

As discussed in U.S. Pat. No. 8,852,136, the entirety of which is incorporated herein by reference, in some embodiments, to ensure proper positioning and functioning of the intraocular shunt, the depth of penetration into the intra-Tenon's adhesion space may be important when performing some methods.

In some methods, the distal tip of the hollow shaft can pierce the sclera and intra-Tenon's adhesion space without coring, removing or causing major tissue distortion of the surrounding eye tissue. The shunt is then deployed from the shaft. Preferably, a distal portion of the hollow shaft (as opposed to the distal tip) completely enters the intra-Tenon's adhesion space before the shunt is deployed from the hollow shaft.

In accordance with some embodiments, the hollow shaft can comprise a flat bevel needle, such as a needle having a triple-ground point. The tip bevel can first pierce through the sclera and into the intra-Tenon's adhesion space by making a horizontal slit. In some methods, the needle can be advanced even further such that the entire flat bevel penetrates into the intra-Tenon's adhesion space, to spread and open the tissue to a full circular diameter.

Further, in accordance with an aspect of some methods, the intra-Tenon's channel can be urged open by the flat bevel portion of the needle so that the material around the opening is sufficiently stretched and a pinching of the shunt in that zone is avoided, thus preventing the shunt from failing due to the pinching or constriction. Full entry of the flat bevel into the intra-Tenon's adhesion space causes minor distortion and trauma to the local area. However, this area ultimately surrounds and conforms to the shunt once the shunt is deployed in the eye.

### Intraocular Shunt Inserters

In some embodiments, the inserter can function as a one-handed device in order to allow an operator to keep their other hand on a fixation device that holds the eye, such as a hook. This can improve surgical control and placement accuracy and makes the surgery easier as well.

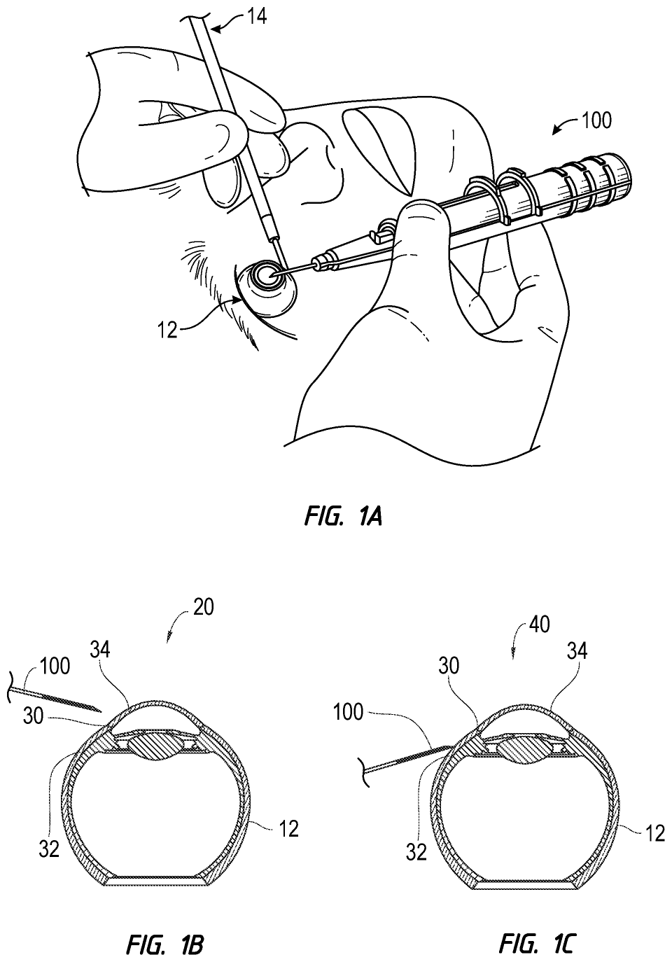

An illustration of a procedure for treating an eye 12 is shown in FIG. 1A. FIG. 1A illustrates the use of an optional hook 14 for holding the eye 12 and an inserter 100 for introducing an intraocular shunt into the eye. FIGS. 1B and IC illustrate general principles of ab interno and ab externo approaches. FIG. 1B is a side, cross-sectional view of an ab interno procedure 20 for implanting an intraocular shunt into an eye 12 using an inserter 100, according to some embodiments. As illustrated, the inserter 100 approaches the eye 12 at a position above or superior to the corneal limbus 30, which generally separates so that sclera 32 of the eye 12 from the cornea 34. As such, in the ab interno procedure, the inserter 100 enters the eye 12 through the cornea 34. Further, FIG. 1C is a side, cross-sectional view of an ab externo procedure 40 for implanting an intraocular shunt into an eye 12 using an inserter 100, according to some embodiments. As illustrated, for the ab externo procedure, the inserter 100 approaches the eye 12 at a position below or inferior to the corneal limbus 30 so that the inserter 100 enters the eye 12 through the sclera 32.

Some embodiments of an intraocular shunt inserter are described herein. The shunt inserter can include a needle defining a lumen. A plunger can be partially disposed within the lumen of the needle. The plunger can advance a shunt through the needle to introduce the intraocular shunt into the eye. These and other features of inserters that can be used in accordance with some embodiments disclosed herein are described in at least Applicant's U.S. Pat. Nos. 6,007,511, 8,721,702, 8,801,766, 8,828,070, 9,095,411, 9,585,790, 10,470,927, 10,085,884, and 10,952,898, and U.S. Patent Pub. No. 2019/0274881, the entireties of each of which are incorporated herein by reference.

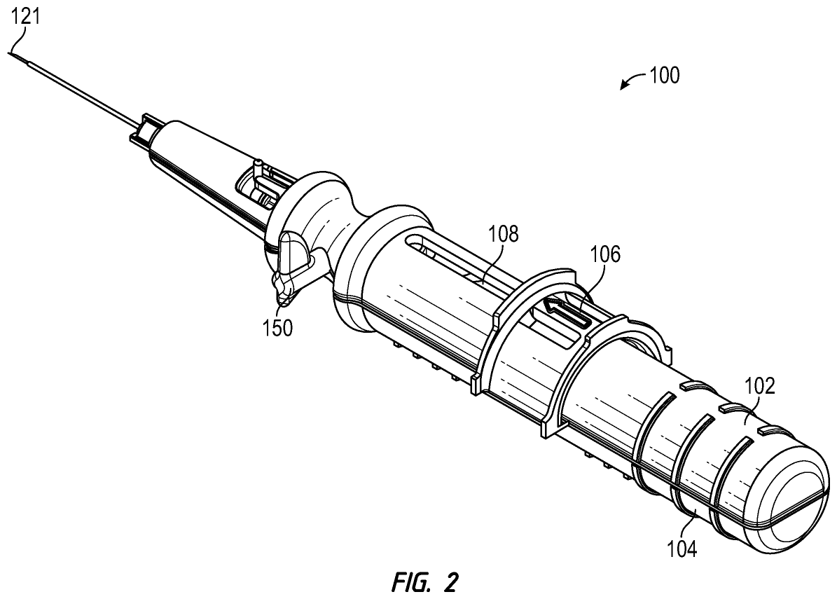

FIGS. 2-4 illustrate details of the inserter 100 shown in FIG. 1. The inserter 100 can be actuated using a single hand, thus facilitating use of the inserter by an operator. The inserter 100 can comprise an upper housing 102, a lower housing 104, a slider component 106, and a needle 121. As shown in FIG. 2, the inserter 100 can be configured such that the slider component 106 is slidable along an elongate slot 108 of the housing 102. The slider component 106 can be selectively movable by an operator in order to actuate movement of components of the inserter 100.

For example, when the slider component 106 moves distally along the slot 108 (i.e., in a direction toward the needle 121), the slider component 106 can result in or cause a shunt (not shown) to be advanced within the needle 121, and in some embodiments, released from the needle 121. In accordance with some embodiments discussed further herein, movement of the slider component 106 can result in translational and/or rotational movement of components of the inserter 100. The sliding movement of the slider component 106 can be converted into rotational movement, which can thereafter be converted to movement along a longitudinal axis of the inserter 100. One of the benefits of this innovative and complex movement-conversion mechanism is that it enables embodiments of the inserter to provide precise, measured movements of its components within a compact assembly.

In the depicted example, the inserter 100 can be configured for ab externo procedures or ab interno procedures by rotating the removable key 150 prior to an implantation procedure. As described herein, the removable key 150 can be positioned in a vertical position (as shown in FIG. 2) to position the shunt relative to the needle 121 for ab externo procedures. Similarly, the removable key 150 can be rotated or otherwise positioned in a horizontal position to move the shunt relative to the needle 121 for ab interno procedures.

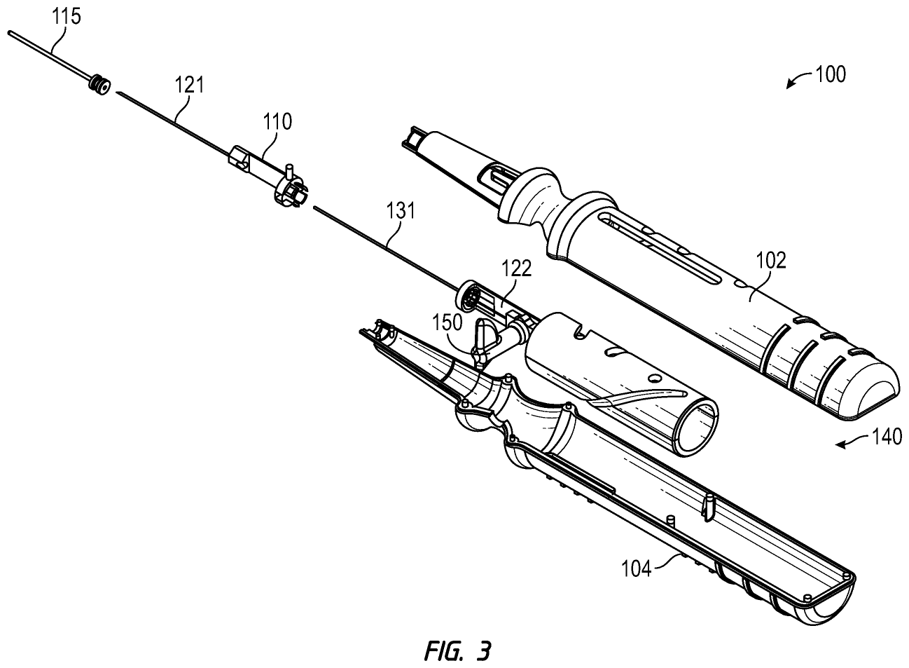

As illustrated in FIG. 3, a plunger 131 can be disposed within the needle 121 to advance the shunt for implantation. The plunger 131 can be slidably movable within a lumen of the needle 121 along a longitudinal axis of the inserter 100. Further, the needle 121 can be slidably movable within a lumen of the sleeve component 115 along the longitudinal axis. In some embodiments, the needle 121 can be attached to a movable needle base 110.

Each of the needle 121 and the plunger 131 can be coupled to respective drive components of a drive assembly 140 disposed within the housing 102 and 104. When in the assembled state, the inserter 100 can be configured such that the needle 121, the plunger 131, and the sleeve component 115 are aligned along or coaxial with the longitudinal axis. Some drive assemblies for actuating a plunger and for withdrawing a needle of an inserter are disclosed in U.S. Pat. Nos. 8,663,303, 8,721,702, 8,758,290, 8,974,511, 9,585,790, 9,636,254, 10,470,927, 10,085,884, and 10,952,898, and U.S. Patent Pub. No. 2019/0274881, the entireties of which are incorporated herein by reference.

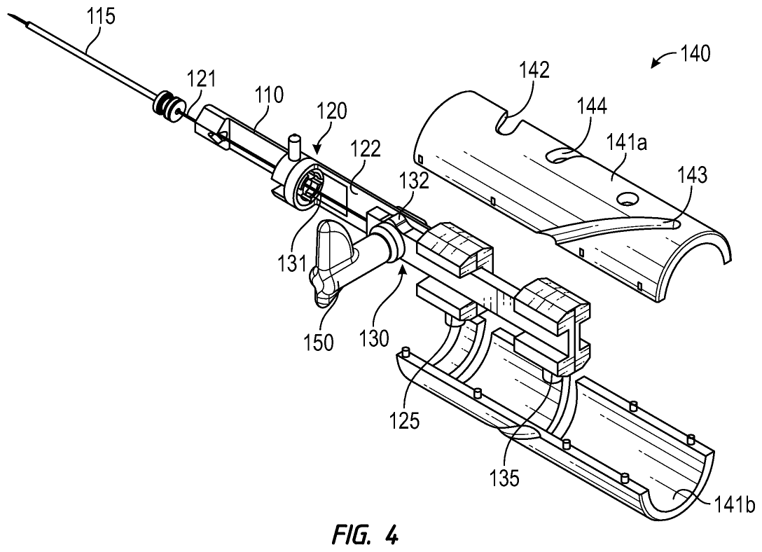

Referring to FIGS. 3 and 4, the needle 121, the plunger 131, and the sleeve component 115 can be operably coupled to the drive assembly 140 and/or the housing 102. For example, a needle assembly 120 can operably couple the needle 121 to the drive assembly 140. The needle assembly 120 can include the needle 121, a needle base 110 and a needle driver 122. In the depicted example, the needle 121 can be coupled to the needle base 110. The needle base 110 can be fixedly coupled to a proximal end portion of the needle 121 such that rotational and longitudinal movement between the needle 121 and the needle base 110 is restricted or prevented. The needle base 110 can be enclosed within a distal end portion of the housing 102 when the inserter 100 is assembled. Further, as illustrated in FIG. 4 and discussed further below, the needle base 110 can be coupled to the drive assembly 140 via the needle driver 122.

Further, as shown in FIG. 4, a plunger assembly 130 can operably couple the plunger 131 to the drive assembly 140. The plunger assembly 130 can include the plunger 131 and a plunger driver 132. The plunger driver 132, can be fixedly coupled to a proximal end portion or midsection of the plunger 131 to restrict or prevent rotational and longitudinal movement of the plunger 131 relative to the plunger driver 132. Further, as illustrated in FIG. 4 and discussed further below, the plunger driver 132 can be coupled to the drive assembly 140.

Furthermore, the sleeve component 115 can be coupled to a portion of the housing 102 and 104. The sleeve component 115 can be coupled so as to prevent rotational and longitudinal movement between the sleeve component 115 and the housing 102 and 104.

As noted above, the needle 121 and the plunger 131 can be operably coupled to the drive assembly 140. Such coupling can occur via the needle driver 122 and the plunger driver 132. In turn, the needle driver 122 and the plunger driver 132 can be coupled to one or more drive components that engage with the drive assembly 140 to the housing 102.

In accordance with some embodiments, the drive assembly 140 can be coupled to the needle 121 and the plunger 131 to actuate movement along the longitudinal axis of the needle 121 and the plunger 131 relative to the housing 102 and 104. For example, the drive assembly 140 can be configured to rotate or slide within the housing 102. The drive assembly 140 can transfer a longitudinal or axial force along the longitudinal axis to the needle 121 and/or the plunger 131, independently or at the same time, to result in movement of the needle 121 and the plunger 131 relative to the housing 102 and 104 along the longitudinal axis.

As discussed herein, motion of the slider component 106 can result in motion of the drive assembly 140 and thereby result in motion of components of the drive assembly 140 relative to the housing 102 and 104. Some embodiments can be configured such that the slider component 106 can be longitudinally movable or slidable along the longitudinal axis relative to the housing 102 and 104 in order to drive or result in linear motion of the needle 121 and the plunger 131 and consequently a shunt.

As shown in FIG. 4, the drive assembly 140 can comprise drive components 141a and 141b configured to engage with the needle driver 122 and plunger driver 132. In some embodiments, longitudinal or linear motion of the slider component 106 along the longitudinal axis can be converted to result in rotation of the drive components 141a and 141b of the drive assembly 140, which can then be converted to result in longitudinal or linear motion of the needle 121 and the plunger 131 along the longitudinal axis relative to the housing 102 and 104. In accordance with some embodiments, motion of the components along the longitudinal axis can be parallel relative to the longitudinal axis.

FIG. 4 also illustrates an embodiment of the drive components 141a and 141b. The drive components 141a and 141b can comprise a groove 143 that can be configured to engage with a corresponding protrusion (not shown) of the slider component 106. Further, the drive components 141a and 141b can also comprise first and second driving grooves 142, 144 that can be configured to slidingly engage corresponding protrusions of the needle driver 122 and the plunger driver 132. Thus, the slider component 106 can comprise a protrusion, the needle driver 122 can comprise a protrusion 125, and the plunger driver 132 can comprise a protrusion 135. This arrangement of slots and protrusions can facilitate the transfer of motion from the slider component 106 to the respective ones of the needle 121 and the plunger 131. Further, the plunger driver 132 and the needle driver 122 can comprise rounded bodies that contact and slide against an inner guide surface of the drive components 141a and 141b when seated within the drive components 141a and 141b.

### The XEN63 System

As noted above, Applicant has successfully commercialized the XEN45 shunt, which provides excellent results and efficacy as a trusted glaucoma treatment option for surgeons. An aspect of at least some of the embodiments disclosed herein is the realization that modifying the XEN45 shunt, along with the inserter through which the XEN45 shunt is delivered, may improve the efficacy of glaucoma treatment and reduce adverse events associated with the treatment.

Especially considering that the response or behavior of the human eye is not always predictable or well understood by surgeons, modifications to any procedures, implants, or inserters do not provide predictable results that are what surgeons anticipate. Moreover, the existing XEN45 shunt has a substantial degree of trust in the industry, given the benefits and efficacy that it provides. Therefore, Applicant's research and development team's decision to change from the 45 μm inner diameter XEN45 shunt has required not only extensive time and evaluation, but also embracing contrarian thought and unproven concepts, as discussed further herein.

Accordingly, some embodiments provide an intraocular shunt and delivery system that improves the reduction of IOP, reduces the reliance on ocular hypotensive drugs, and minimizes postoperative needling to maintain the reduced IOP. Such benefits and advantages can be achieved by delivering a XEN45 shunt that has been modified to have a 63 μm inner diameter (i.e., the XEN63 shunt), using an inserter that was modified to have a 27 Gauge needle (referred to collectively as the XEN63 system). One of the benefits of using a modified 27 Gauge needle is that it enables physicians to deliver the XEN63 shunt using an inserter with a smaller gauge needle than what would otherwise be required (e.g., 25 Gauge needle) in view of the larger diameter of the XEN63 shunt. The 27 Gauge needle permits patients to benefit from the larger lumen of the XEN63 shunt while experiencing a lower likelihood of adverse events due to the XEN63 shunt being delivered via a smaller, modified 27 Gauge needle (e.g., versus a larger needle, such as a 25 Gauge needle, which was used for such a larger-lumen shunt like the XEN63 shunt). As illustrated through the evaluations and study discussed further below, an aspect of at least some embodiments disclosed herein relates to the realization that the XEN63 system provides surprising, unexpected benefits that are superior to prior systems or devices. For example, the collective effect of delivering the XEN63 shunt in combination with an inserter with a 27 Gauge needle provides an immediate, clear, and substantial reduction in IOP, and surprisingly, without a corresponding increase in risk of hypotony compared to the XEN45 shunt, as well as reducing the need for postoperative needling and the use of hypotensive antiglaucoma medications.

In some embodiments, the XEN63 shunt can be manufactured in a manner that reduces the acceptable tolerance for the XEN63 shunt's outer diameter compared to conventional shunt manufacturing procedures. In some embodiments, the XEN63 shunt can have an outer diameter of about 170 μm to about 280 μm, about 170 μm, about 180 μm, about 190 μm, about 200 μm, about 210 μm, about 220 μm, about 230 μm, about 240 μm, about 250 μm, about 260 μm, about 270 μm, about 280 μm, or in some embodiments, about 240 μm to about 260 μm. The XEN63 shunt can define at least one interior flow path having a shunt inner diameter of between about 50 μm and about 70 μm, such as about 55 μm, 60 μm, 65 μm, or in some embodiments, about 63 μm.

In some embodiments, the XEN63 shunt can expand to a maximum outer diameter of about 260 μm. Further, the XEN63 shunt can have a shunt wall thickness of between about 80 μm and about 110 μm, about 85 μm and about 105 μm, or about 88 μm to about 99 μm. Furthermore, the XEN63 shunt can have a shunt length of about 5 mm, about 6 mm, or about 7 mm.

The inserter used in the XEN63 system can comprise any of the features of the inserters discussed above. In accordance with some embodiments, the inserter used to deliver the XEN63 shunt can comprise a 27 Gauge needle, which provides a nominal inner diameter of between about 210 μm to about 280 μm, between about 230 μm to about 270 μm, or about 260 μm. The nominal outer diameter of the needle can be about 400 μm to about 420 μm, such as about 410 μm, further, the needle can have a wall thickness of between about 70 μm and about 80 μm.

### The XEN63 System Evaluation Surveys

During the evaluation of the XEN63 system, surgeons participating in the evaluation filled out three surveys at different time points: a pre-evaluation survey at the start of the evaluation, before they have completed any XEN63 implantation procedures; an early experience survey, after completing three to four XEN63 shunt implantation procedures; and a final evaluation survey, completed about three to four months after implanting their first XEN63 shunt.

In the pre-evaluation survey, surgeons provided responses to a variety of questions relating to their experience to date with the XEN45 shunt and their expectations for the XEN63 shunt delivery. The pre-evaluation survey took place before any XEN63 shunts were implanted. Consistently, across the group, surgeons had significant concerns about the larger lumen of the XEN63 shunt. While the surgeons expected that the larger lumen would achieve a lower IOP, they also expected that it would result in more frequent hypotony-associated complications than those experienced using the XEN45 shunt. Further, surgeons were pessimistic about the effectiveness of the XEN63 shunt, indicating that they expected patients receiving the XEN63 shunt would require more postoperative IOP-lowering medication compared to those that had received the XEN45 shunt. In addition, surgeons also believed that patients receiving the XEN63 shunt would require a higher needling rate compared to those patients receiving the XEN45 shunt.

Next, in the early experience survey, after completing several XEN63 shunt implantation procedures, surgeons remarked that by an “Immediate Day 1,” the XEN63 shunt resulted in a lower IOP compared to XEN45 shunt. At this point, 48% of the surgeons also remarked that their experience with the XEN63 shunt was better than expected, while another 48% of the surgeons noted that their experience with the XEN63 shunt was as expected.

Finally, in the post- or final evaluation survey, conducted about three to four months after implanting their first XEN63 shunt, surgeons remarked that their experience with the XEN63 shunt, implanted using an inserter having a 27 Gauge needle (the procedure collectively being referred to as the “XEN63 system”), was better than expected. The surgeons remarked that the XEN63 shunt is implanted in the same way, requiring about the same time as the XEN45 shunt. These surgeons noted that the XEN63 system provides immediate lowering of TOP, compared to the XEN45 shunt. The surgeons also noted that when successful needling was performed, the restoration of the bleb was more obvious for the XEN63 shunt than for the XEN45 shunt. The surgeons were more likely to consider the XEN63 system for combined phaco/XEN63 shunt procedures than the XEN45 shunt. The surgeons believed the XEN63 shunt could completely replace the XEN45 shunt, at least for primary usage. Indeed, 63% of the surgeons also indicated that compared with the XEN45 shunt, the XEN63 system would require less postoperative needling.

Overall, in the post- or final evaluation survey, the surgeons indicated that prospect of being able to use the XEN63 system, given the larger lumen of the XEN63 shunt, is appealing. The surgeons believed that XEN63 system is a promising option for achieving potentially lower IOPs values and with less need for pressure-lowering medications compared to those required to be used with the XEN45 shunt. Indeed, compared with the XEN45 shunt, the surgeons believed that the XEN63 shunt would be more appropriate. In fact, 13% of the surgeons indicated that the XEN63 system would completely replace the XEN45 shunt for future work in all patients, and another 75% indicated that they would primarily use the XEN63 system in patients in the future.

Accordingly, the evaluation surveys indicate that not only was there an initial high degree of skepticism specific to the basic viability of using the XEN63 system due to hypotony and needling concerns, but there was also a later, profound shift in the surgeon's views about the effectiveness and long-term viability of the XEN63 system. As noted above, an overwhelming majority of the surgeons indicated that not only would the XEN63 system be the primary choice for their patient work, but many indicated that it would also completely replace the XEN45 shunt for future work in all patients.

Thus, medical experts in this field initially indicated that the XEN63 system, e.g., the implantation of the XEN63 shunt using an inserter with a 27 Gauge needle, was a surgical option that they would avoid. However, upon trial and observation, the vast majority of surgeons were surprised to find that the XEN63 system was the new best way for them to conduct effective, safe, and successful glaucoma treatments. These experiential results themselves provide convincing evidence not only of the surprising attributes of the solution provided by the embodiments of the XEN63 system disclosed herein, including its numerous benefits and advantages, but also of the viability and expected commercial success of the XEN63 system (given that these same medical experts are representative of the doctors that will be recommending a type of surgery to their patients). The new features and configuration of the XEN63 system not only went against conventional wisdom and went against accepted practices (as evidenced by the statements and surveys from medical experts), but these medical experts and the prior art have not recognized that these features and configurations could be modified to provide an intraocular shunt and delivery system that improves the reduction of TOP, reduces the reliance on ocular hypotensive drugs, and minimizes postoperative needling to maintain the reduced IOP as much as those disclosed herein, such as by delivering a shunt having a 63 μm inner diameter using a 27 Gauge needle.

### The XEN63 System Study

In addition to the various surveys conducted by Applicant regarding embodiments of the XEN63 system disclosed herein, Applicant arranged for a study to be conducted by doctors using open-angle glaucoma (“OAG”) patients who received a XEN63 shunt, either alone or in combination with cataract surgery, between February and June 2020. Applicant did not participate in the data analysis or redaction of the manuscript. The authors of the study concluded that use of the XEN63 system (the XEN63 shunt being implanted using an inserter having a 27 Gauge needle), significantly lowered IOP and reduced the number of antiglaucoma medications, with a good short-term safety profile. These conclusions were supported by further data and consistent through 18 months of follow-up.

Participants were required to meet the following inclusion criteria: age ≥40 years, clinical diagnosis of OAG, and an unmet target IOP despite medical therapy. Patients with narrow-angle glaucoma (unless the surgeon believed that a sufficient space allowed to safely implant the device), severe conjunctival scars, ocular pemphigoid, phacodonesis, progressive retinal or optic nerve disease of any cause, or history of major ocular surgery (except phacoemulsification) within the previous 6 months were excluded of the study. In the study, a MIGS device, the XEN63 shunt (made by Allergan, Irvine, CA, US) was used. The XEN63 shunt used was composed of porcine gelatin crosslinked with glutaraldehyde. The stent was 6 mm in length, with an outer diameter of 180 μm and an inner diameter of 63 μm. All the surgical procedures were performed, under local anesthesia, by the same two experienced surgeons.

At baseline, each subject underwent a standard ophthalmic exam, which included a detailed medical history, anterior segment and fundus examination, best corrected visual acuity (“BCVA”), IOP measurement assessed at 9 am (±1 hour) using Goldmann applanation tonometry, and gonioscopy. A computerized visual field (Humphrey visual field analyzer; Carl Zeiss Meditec, Dublin, CA) was performed as the baseline examination within six months before implantation of the XEN63 shunt. Further, follow-up visits included anterior segment examination, paying special attention to filtering bleb; BCVA; TOP; dilated fundus examination; and the incidence of adverse events.

Topical and systemic IOP-lowering medications were suspended on the day of surgery. Patients with bleb fibrosis, flat bleb, and/or elevated IOP underwent needling, which was performed in the theater.

The XEN63 shunt was placed in the superior nasal quadrant using a standard ab interno technique. See Fea A M, Bron A M, Economou M A, Laffi G. Martini E, Figus M, et al. European study of the efficacy of a cross-linked gel stent for the treatment of glaucoma. J Cataract Refract Surg. 2020; 46(3):441-450. doi: 10.1097/j.jcrs.0000000000000065; Fea A M, Durr G M, Marolo P, Malinverni L, Economou M A, Ahmed I. XEN® Gel Stent: A Comprehensive Review on Its Use as a Treatment Option for Refractory Glaucoma. Clin Ophthalmol. 2020; 14:1805-1832. doi: 10.2147/OPTH.S178348; and Femindez-Garcia A, Zhou Y, Garcia-Alonso M, Andrango H D, Poyales F, Garzon 19 N. Comparing Medium-Term Clinical Outcomes following XEN® 45 and XEN® 63 Device Implantation. J Ophthalmol. 2020:2020:4796548. doi: 10.1155/2020/4796548, the entireties of each of which are incorporated herein by reference. After anesthesia and skin disinfection, conjunctival upper-nasal quadrant was marked 3 mm from the limbus. Before surgery, 0.1 ml of MMC 0.02%-0.03% was injected intra-tenon in the supero-nasal quadrant.

After injecting a viscoelastic with high cohesivity, the pre-loaded injector needle was inserted at the inferotemporal quadrant through a 1.8 mm corneal paracentesis. An intraoperative goniolens was used to verify placement through the scleral spur. Once the goniolens was removed, the tip of the inserter needle advanced, approximately 3 mm through the sclera, and the XEN63 shunt was finally positioned into the subconjunctival space. The position of the XEN63 shunt in the anterior chamber was checked, by gonioscopy, before removing viscoelastic. In order to confirm the lack of adhesions, sideways movements of the XEN63 shunt were performed until it moved freely under the conjunctiva.

Afterwards, the function and bleb formation of the XEN63 shunt were assessed by constant irrigation with balanced salt solution (BSS). Finally, the corneal incisions were hydrated with BSS.

Perioperative care included antibiotic therapy four times a day during the first week and anti-inflammatory therapy with steroids was administered six times a day, which was slowly tapered over three months.

In eyes that underwent cataract surgery, phacoemulsification was performed using the surgeon's preferred technique, and the XEN63 shunt was implanted in all cases after cataract surgery.

Special attention was also paid to avoid or delay ocular hypotensive drug reintroduction as much as possible. Before starting any postoperative antiglaucoma medications, surgeons performed either needling or bleb revision. If this approach failed or patient refused to undergo these procedures, topical hypotensive medication was reintroduced.

### The XEN63 System Study Outcomes and Analysis

The outcomes of the XEN63 study were assessed using primary endpoints of (i) the IOP at month 3 and (ii) the incidence of serious adverse events. Secondary endpoints included: (iii) incidence of any adverse event; (iv) reduction in number of ocular hypotensive medications from baseline to month 3; (v) proportion of patients achieving an IOP lowering ≥30% and ≥20% without antiglaucoma medications; (vi) proportion of patients achieving a final IOP≤12 mm Hg; ≤14 mm Hg; ≤16 mm Hg; or ≤18 mm Hg without medications; and (vii) incidence of non-serious adverse events.

A standard statistical analysis was performed using Prism 9 version 9.0 (GraphPad Software; San Diego, CA 92108, USA). Although sample size was not calculated before the study, a post hoc analysis was conducted for testing the adequacy of sample. The post hoc power analyses was determined for an alpha level of 0.05, the study sample size, and the effect size observed in the study. See Yuan K-H, Maxwell S. On the Post Hoc Power in Testing Mean Differences. Journal of Educational and Behavioral Statistics. 2005; 30(2):141-167. doi:10.3102/10769986030002141, the entirety of which is incorporated herein by reference. Data are expressed as number (percentage), mean t standard deviation (SD), or mean (95% confidence interval, CI) as appropriate. Data were tested for normal distribution using a Shapiro-Wilks test.

Changes in IOP and number of ocular hypotensive medications were performed by means of repeated measures ANOVA and the Greenhouse-Geisser correction test. The last-observation-carried-forward method was used to impute missing data. P value of less than 0.05 was considered significant.

### Results of the XEN63 System Study

As illustrated in Table 1 below, overall, twenty-three patients met the inclusion/exclusion criteria requirements for the study, with 20 having received a treatment in which only the XEN63 shunt was implanted and another three receiving a treatment in which the XEN63 shunt along with a phacoemulsification or cataract procedure. The mean age was 67.8±15.3 years and 15 (65.2%) were women. Table 1 also shows the main baseline clinical and demographic characteristics of the study population.

TABLE 1
Main Baseline Clinical and Demographic
Characteristics of the Study Sample

| | Overall | XEN63 | Phaco + |
|---|---|---|---|
| Variable | (n = 23) | (n = 20) | XEN63 (n = 3) |
| Age, years | | | |
| Mean = SD | 67.8 ± 15.3 | 67.3 ± 15.9 | 71.3 ± 12.4 |
| Sex, n (%) | | | |
| Women Men | 15 (65.2) | 14 (70.0) | 1 (33.3) |
| | 8 (34.8) | 6 (30.0) | 2 (66.7) |
| Type of | | | |
| glaucoma | | | |
| POAG | 14 (60.9) | 12 (60.0) | 2 (66.7) |
| Uveitic PXG | 4 (17.4) | 3 (15.0) | 1 (33.3) |
| PACG | 1 (4.3) | 1 (5.0) | 0 (0.0) |
| Traumatic | 1 (4.3) | 1 (5.0) | 0 (0.0) |
| | 1 (4.3) | 1 (5.0) | 0 (0.0) |
| Missing | 2 (8.7) | 2 (10.0) | 0 (0.0) |
| information | | | |
| Previous | 21 (91.3) | 18 (90.0) | 3 (100.0) |
| laser, n | | | |
| (%) | | | |
| No SLT | 1 (4.3) | 1 (5.0) | 0 (0.0) |
| Nd:YAG | 1 (4.3) | 1 (5.0) | 0 (0.0) |
| Iridotomy | | | |
| Previous | 2 (8.7) | 0 (0.0) | 3 (100.0) |
| surgery*, | | | |
| n (%) None | | | |
| Cataract | 14 (60.9) | 14 (70.0) | 0 (0.0) |
| Refractive | 3 (13.0) | 3 (15.0) | 0 (0.0) |
| (laser) | | | |
| Trabecular | 2 (8.7) | 2 (10.0) | 0 (0.0) |
| MIG | | | |
| Subconjunctival | 2 (8.7) | 2 (10.0) | 0 (0.0) |
| MIG | | | |
| BCVA, ETDRS | 0.49 ± 0.26 | 0.54 ± 0.24 | 0.23 ± 0.15 |
| Mean ± SD | | | |
| ECC | 2217.9 ± 343.1 | 2223.4 ± 297.0 | 2161.0 ± 563.8 |
| Mean ± SD | | | |
| MD, dB | −17.03 ± 9.96 | −16.71 ± 10.51 | −18.23 ± 9.44 |
| Mean ± SD | | | |
| PSD, dB | 7.00 ± 2.28 | 7.10 ± 3.08 | 6.66 ± 3.2 |
| Mean ± SD | | | |
| NTOHM | 2.27 ± 0.94 | 2.26 ± 0.99 | 2.33 ± 0.58 |
| Mean ± SD | | | |
| IOP, mm Hg | 28.7 ± 6.44 | 28.4 ± 6.81 | 30.3 ± 3.2 |
| Mean ± SD | | | |

*Patients may have undergone more than one procedure.
Abbreviations:
Phaco: Phacoemulsification;
SD: Standard deviation;
POAG: Primary open-angle glaucoma;
PXG: Pseudoexfolitive glaucoma;
PACG: Primary angle-closure glaucoma;
SLT: Selective laser trabeculoplasty;
YAG: Neodymium-doped Yttrium Aluminium Garnet;
MIG: Minimally invasive glaucoma device;
BCVA: Best corrected visual acuity;
ETDRS: Early treatment diabetic retinopathy study;
ECC: Endothelial cell count;
MD: Mean defect;
PSD: Pattern standard deviation;
NTOHM: Number of topical ocular hypotensive medications;
IOP: Intraocular pressure.

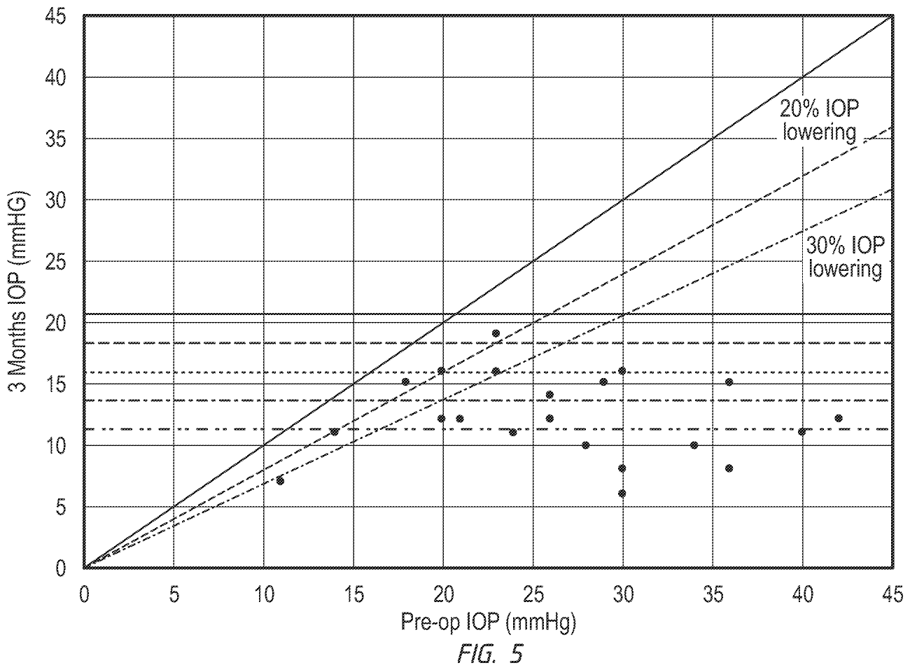

The results are also summarized and represented in FIG. 5, which is a scatter plot of the IOP at baseline and month 3. The scatter plot uses the following information: the mean difference −14.8±6.0 mm Hg, 95 Confidence interval −18.4 to −11.2 mm Hg; p<0.0001 (two-tailed paired-samples Student t test).

As illustrated by FIG. 5, the 20% and the 30% lines indicate the level beneath which an IOP reduction of more than 20% or 30%, respectively, compared to baseline value before surgery, was reached by the individual cases. Further, in the overall study population, baseline TOP was significantly reduced from 27.0±7.8 mm Hg to 12.2±3.4 mm Hg at month 3, p<0.0001, and consistent through 18 months of follow-up.

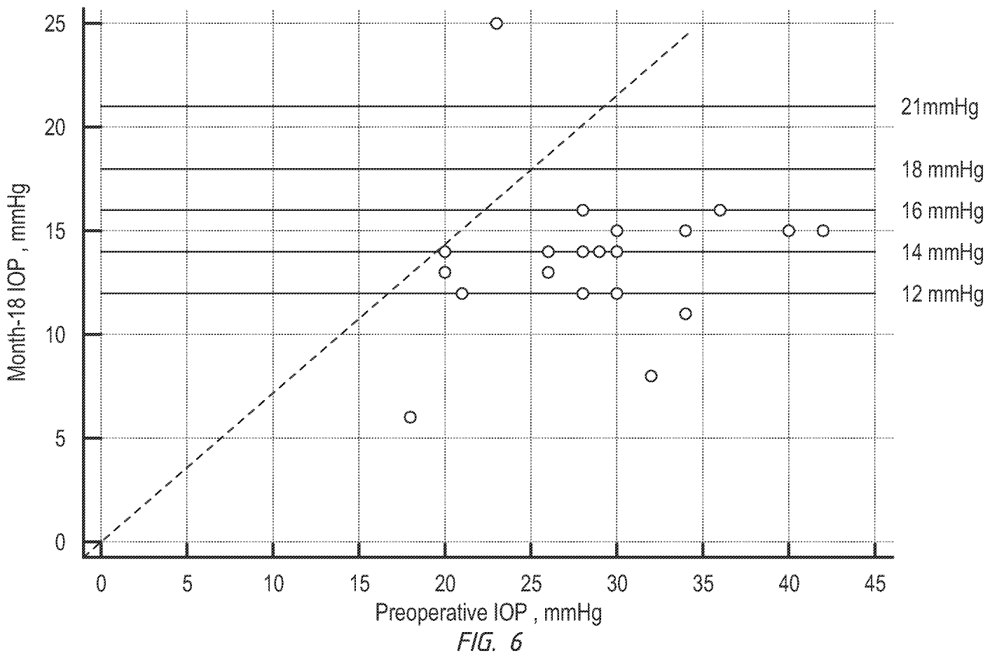

Referring to FIG. 6, results are summarized and represented by a scatter plot of the IOP at baseline and month 18. Data were plotted from the baseline IOP on the X-axis and the month 18 visit IOP on the Y-axis to determine an overall visual assessment. Similar to the results illustrated in FIG. 5 for month 3, by month 18, preoperative IOP was significantly reduced from 26.5±8.2 mmHg and 30.3±3.3 mmHg to 14.2±3.7 mmHg and 13.7±1.5 mmHg in the XEN alone and XEN+phacoemulsification groups, respectively (p<0.0001 and p=0.0012, respectively). The baseline and month 18 IOP had a mean difference of −12.9 mmHg; 95 CI: −16.9 to −8.9 mmHg; p<0.0001 (repeated measures ANOVA and the Greenhouse-Geisser correction test).

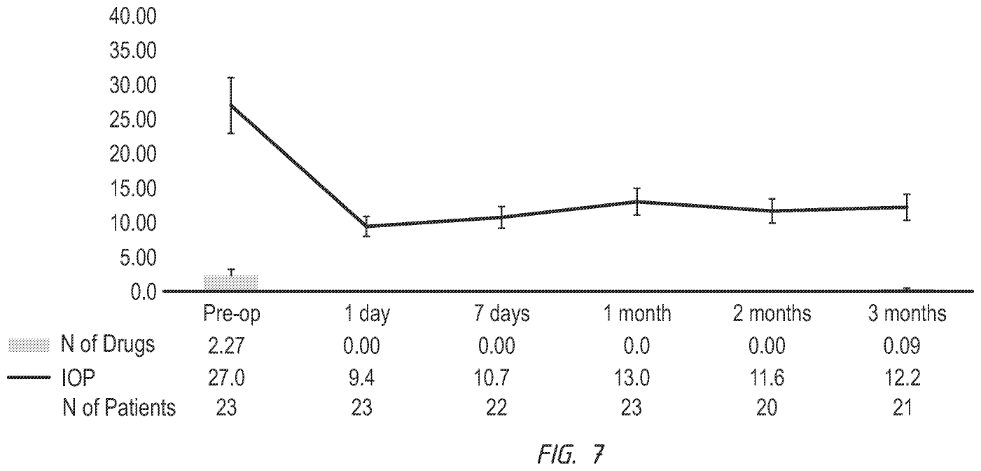

Next, FIG. 7 shows the mean IOP over the course of the study follow-up through month 3. FIG. 7 is an overview of the mean IOP and number of hypotensive medications over the course of the study follow-up in the overall study population. In FIG. 6, the vertical bars represent standard deviation. Further, when compared to baseline, mean (95% confidence interval) IOP lowering was −17.6 (−22.0 to −13.1) mm Hg, p<0.0001; −16.3 (−20.8 to −11.8) mm Hg, p<0.0001; −14.0 (−18.6 to −9.4) mm Hg, p<0.0001; −15.4 (−20.2 to −10.6) mm Hg, p<0.0001; and −14.8 (−20.1 to −9.5) mm Hg, p<0.0001 at day 1, day 7, and months 1, 2, and 3, respectively. At month three, mean lowering IOP was 40.8±23.5%, with 14 eyes (60.9%) and 16 eyes (69.6%) achieving an IOP lowering ≥30% and ≥20% without hypotensive medication, respectively.

The mean number of topical ocular hypotensive medications was significantly reduced from 2.27±0.94 drugs at baseline to 0.09±0.42 drugs at month 3, p<0.0001. At month 3, 22 eyes (95.7%) did not receive any antiglaucoma medication. Additionally, the BCVA did not change over the course of the study (mean change: 0.1±0.2).

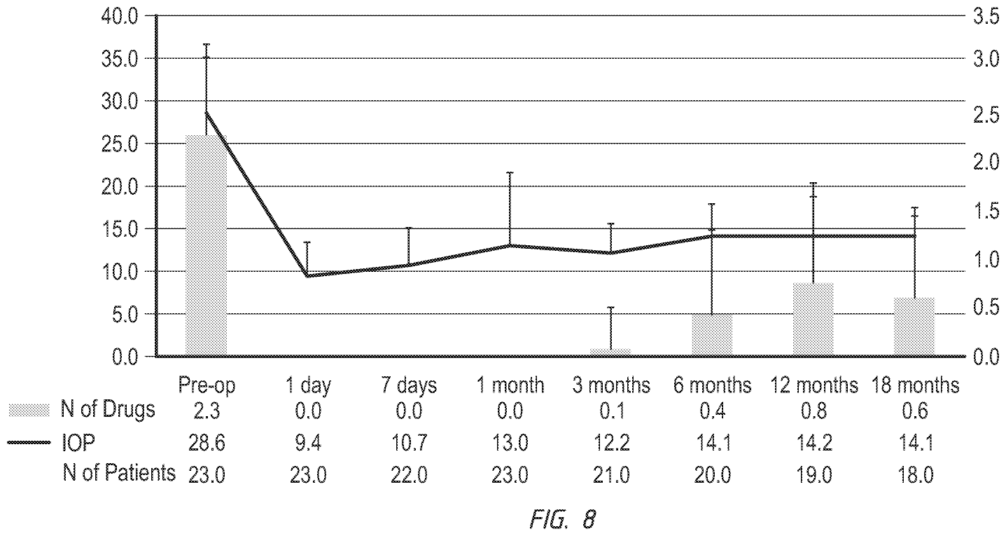

Referring now to FIG. 8, the mean IOP data was consistent through eighteen months of follow-up. In the overall study sample, preoperative IOP was significantly lowered from 27.0±7.8 mmHg to 14.1±3.4 mmHg at month 18 (mean difference −12.9 mmHg; 95 CI: −16.9 to −8.9 mmHg; p<0.0001), as shown in FIG. 8.

Referring now to Table 2 below, at day 7, four eyes (17.4%) showed a ≥2-line worsening in BCVA as compared to baseline; while four eyes (17.4%) showed a ≥2-line improvement as compared to baseline (table 2). The number of patients at month 18 were less than the number at month 3 due to attrition.

TABLE 2
Overview of the number (%) of patients who experienced changes in best
corrected visual acuity throughout the study.

| Change in Best-Corrected | | | Month | Month | Month |
|---|---|---|---|---|---|
| Visual Acuity (BCVA) | Day 1 | Day 7 | 1 | 3 | 18 |
| Worse ≥ 2 lines, n (%) | 4 (17.4) | 4 (17.4) | 2 (8.7) | 2 (8.7) | 1 (5.6) |
| Worse ≥ 1 line, n (%) | 6 (26.1) | 7 (30.4) | 3 (13.0) | 3 (13.0) | 3 (16.7) |
| Unchanged, n (%) | 9 (39.1) | 6 (26.1) | 8 (34.8) | 6 (26.1) | 2 (11.1) |
| Improvement ≥ | 0 (0.0) | 2 (8.7) | 3 (13.0) | 2 (8.7) | 4 (22.2) |
| 1 line, n (%) | | | | | |
| Improvement ≥ 2 | 4 (17.4) | 4 (17.4) | 7 (30.4) | 8 (34.8) | 5 (27.8) |
| lines, n (%) | | | | | |

In addition, regarding safety, four eyes (17.4%) had hypotony (an IOP≤6 mm Hg) at postoperative day one, which was successfully resolved without sequelae. Three eyes (13.0%) required needling over the course of the study follow-up (mean time for needling 35.6±9.7 days), one eye with mitomycin-c and two ones with 5-fluorouracil. Only one eye underwent needling due to elevated IOP. Five eyes (21.7%) underwent digital ocular massage. One eye (4.3%) had anterior chamber bleeding during the surgery, one eye (4.3%) had a 1.5 mm hyphema at day 1, and four eyes (17.4%) had choroidal detachment (three at day 7 and 1 at day 15), which was successfully resolved with medical treatment, at month-1 visit.

At month 18, regarding safety, four (17.4%) eyes had hypotony (defined as an IOP 56 mm Hg) at postoperative day 1, which was successfully resolved without sequalae. One (4.3%) eye had anterior chamber bleeding during the surgery, one (4.3%) eye had a 1.5 mm hyphema at postoperative day 1, and four (17.4%) had choroidal detachment (three at postoperative day 7 and one at postoperative day 15), which was successfully resolved with medical treatment, at month 1 visit. Four (17.4%) eyes underwent a needling procedure (mean time for needling 42.9±11.2 days), although only one eye did so due to an elevated IOP. Four eyes underwent additional surgeries, two (8.7%) eyes underwent trabeculectomy, one (4.3%) eye underwent a device replacement (a new XEN45 device was implanted), and one eye (4.3%) underwent a high-intensity focused ultrasound cyclodestruction (HIFU) procedure (Table 3). However, according to the last follow-up visit, another patient needed either a needling or additional surgery.

### Discussion of the XEN63 System Study

According to the results of the collaborative initial glaucoma treatment study (“CIGTS”) (see Jampel H D, Musch D C, Gillespie B W, Lichter P R, Wright M M, Guire K E; Collaborative Initial Glaucoma Treatment Study Group. Perioperative complications of trabeculectomy in the collaborative initial glaucoma treatment study (CIGTS). Am J Ophthalmol. 2005; 140(1):16-22. doi: 10.1016/j.ajo.2005.02.013, the entirety of which is incorporated herein by reference), trabeculectomy was associated with a fifty percent incidence of early postoperative complications. In the same study, choroidal detachment, anterior chamber bleeding, or anterior chamber flattening had an incidence equal to or greater than 10%. See Jampel H D, Musch D C, Gillespie B W, Lichter P R, Wright M M, Guire K E; Collaborative Initial Glaucoma Treatment Study Group. Perioperative complications of trabeculectomy in the collaborative initial glaucoma treatment study (CIGTS). Am J Ophthalmol. 2005; 140(1):16-22. doi: 10.1016/j.ajo.2005.02.013, the entirety of which is incorporated herein by reference. Additionally, the results of the tube versus trabeculectomy study showed that the rate of early postoperative complications (those developed within the first month after surgery) was 37% in the trabeculectomy group. See Gedde S J. Hemdon L W, Brandt J D, Budenz D L, Feuer W J, Schiffman J C; Tube Versus Trabeculectomy Study Group. Postoperative complications in the Tube Versus Trabeculectomy (TVT) study during five years of follow-up. Am J Ophthalmol. 2012; 153(5):804-814.e1. doi: 10.1016/j.ajo.2011.10.024, the entirety of which is incorporated herein by reference.

According to the results of the XEN63 study, even if they are limited to only 23 eyes and with short follow-up of 3 months, the XEN63 system, including the delivery using an inserter with a 27 Gauge needle, provided a better IOP lowering effect than the prior XEN45 shunt. Results and discussion of the efficacy of the XEN45 shunt are documented in various publications. See e.g., De Gregorio A. Pedrotti E, Russo L, Morselli S. Minimally invasive combined glaucoma and cataract surgery: clinical results of the smallest ab interno gel stent. Int Ophthalmol. 2018; 38(3):1129-1134. doi: 10.1007/s10792-017-0571-x; Reitsamer H, Sng C. Vera V, Lenzhofer M. Barton K, Stalmans I; Apex Study Group. Two-year results of a multicenter study of the ab interno gelatin implant in medically uncontrolled primary open-angle glaucoma. Graefes Arch Clin Exp Ophthalmol. 2019; 257(5):983-996. doi: 10.1007/s00417-019-04251-z; Chatzara A, Chronopoulou I, Theodossiadis G, Theodossiadis P, Chatziralli I. XEN Implant for Glaucoma Treatment: A Review of the Literature. Semin Ophthalmol. 2019; 34(2):93-97. doi: 10.1080/08820538.2019.1581820; Marcos Parra M T, Salinas López JA, Lopez Grau N S, Ceausescu A M, Perez Santonja J J. XEN implant device versus trabeculectomy, either alone or in combination with phacoemulsification, in open-angle glaucoma patients. Gracfes Arch Clin Exp Ophthalmol. 2019; 257(8):1741-1750. doi: 10.1007/s00417-019-04341-y; Fea A M, Bron A M, Economou M A, Laffr G, Martini E. Figus M, et al. European study of the efficacy of a cross-linked gel stent for the treatment of glaucoma. J Cataract Refract Surg. 2020:46(3):441-450. doi: 10.1097/j.jcrs.0000000000000065; Fea A M, Durr G M, Marolo P, Malinverni L. Economou M A, Ahmed I. XEN<sup>@</sup> Gel Stent: A Comprehensive Review on Its Use as a Treatment Option for Refractory Glaucoma. Clin Ophthalmol. 2020; 14:1805-1832. doi: 10.2147/OPTH.S178348; Grover D S, Flynn W J. Bashford K P, Lewis R A. Duh Y J, Nangia R S, et al. Performance and Safety of a New Ab Intemo Gelatin Stent in Refractory Glaucoma at 12 Months. Am J Ophthalmol. 2017; 183:25-36. doi: 10.1016/j.ajo.2017.07.023; Ibáñez-Muñoz A, Soto-Biforcos V S, Chacćn-González M, Rúa-Galisteo O, Ameta-Los Santos A, Lizuain-Abadia M E, et al. One-year follow-up of the XEN® implant with mitomycin-C in pseudoexfoliative glaucoma patients. Eur J Ophthalmol. 2019; 29(3):309-314. doi: 10.1177/1120672118795063; Laborda-Guirao T, Cubero-Parra J M, Hidalgo-Torres A. Efficacy and safety of XEN 45 gel stent alone or in combination with phacoemulsification in advanced open angle glaucoma patients: 1-year retrospective study. Int J Ophthalmol. 2020; 13(8):1250-1256. doi: 10.18240/ijo.2020.08.11; and Theilig T. Rehak M, Busch C, Bormann C, Schargus M, Unterlauft J D. Comparing the efficacy of trabeculectomy and XEN gel microstent implantation for the treatment of primary open-angle glaucoma: a retrospective monocentric comparative cohort study. Sci Rep. 2020; 10(1):19337. doi: 10.1038/s41598-020-76551-y; the entireties of each of which are incorporated herein by reference.

Additionally, the patients in the study demonstrated a good hypotensive profile, with a mean IOP lowering of 40.8±23.5% and 16 eyes (69.6%) achieving an IOP lowering ≥30% without hypotensive medication.

One of the aspects that makes the study so significant is that it is well-known that the XEN45 shunt has shown a good early and long-term postoperative safety profile, while maintaining good IOP lowering. &e e.g., De Gregorio A, Pedrotti E. Russo L. Morselli S. Minimally invasive combined glaucoma and cataract surgery: clinical results of the smallest ab interno gel stent. Int Ophthalmol. 2018; 38(3):1129-1134. doi: 10.1007/s10792-017-0571-x; Reitsamer H, Sng C. Vera V, Lenzhofer M, Barton K, Stalmans I; Apex Study Group. Two-year results of a multicenter study of the ab interno gelatin implant in medically uncontrolled primary open-angle glaucoma. Graefes Arch Clin Exp Ophthalmol. 2019; 257(5):983-996. doi: 10.1007/s00417-019-04251-z; Chatzara A, Chronopoulou I, Theodossiadis G. Theodossiadis P, Chatziralli I. XEN Implant for Glaucoma Treatment: A Review of the Literature. Semin Ophthalmol. 2019; 34(2):93-97. doi: 10.1080/08820538.2019.1581820; Marcos Parra M T, Salinas López JA, López Grau N S, Ceausescu A M, Pérez Santonja J J. XEN implant device versus trabeculectomy, either alone or in combination with phacoemulsification, in open-angle glaucoma patients. Graefes Arch Clin Exp Ophthalmol. 2019; 257(8):1741-1750. doi: 10.1007/s00417-019-04341-y; Fea A M, Bron A M, Economou M A, Laffi G, Martini E, Figus M. et al. European study of the efficacy of a cross-linked gel stent for the treatment of glaucoma. J Cataract Refract Surg. 2020; 46(3):441-450. doi: 10.1097/j.jcrs.0000000000000065; Fea A M, Durr G M, Marolo P, Malinvemi L, Economou M A, Ahmed I. XEN® Gel Stent: A Comprehensive Review on Its Use as a Treatment Option for Refractory Glaucoma. Clin Ophthalmol. 2020; 14:1805-1832. doi: 10.2147/OPTH.S178348; the entireties of each of which are incorporated herein by reference.

Therefore, when comparing the results achieved using the XEN63 system with other studies, which reported IOP data of XEN45 shunt at month 3, the mean IOP achieved with the XEN63 system was consistently lower, as shown in Table 3 below. The same holds true when the differential reduction in pressure is examined. See Marcos Parra M T, Salinas López JA, López Grau N S, Ceausescu A M, Pdrez Santonja J J. XEN implant device versus trabeculectomy, either alone or in combination with phacoemulsification, in open-angle glaucoma patients. Graefes Arch Clin Exp Ophthalmol. 2019; 257(8):1741-1750. doi: 10.1007/s00417-019-04341-y; and Laborda-Guirao T, Cubero-Parra J M, Hidalgo-Torres A. Efficacy and safety of XEN 45 gel stent alone or in combination with phacoemulsification in advanced open angle glaucoma patients: 1-year retrospective study. Int J Ophthalmol. 2020; 13(8):1250-1256. doi: 10.18240/ijo.2020.08.11; the entireties of each of which are incorporated herein by reference

TABLE 3
Overview of the Intraocular Pressure (IOP) and Number of Hypotensive Medications in Eyes
Underwent XEN45 Shunt Surgery in Different Studies

| | Baseline | | IOP | IOP | Mean | Mean |
|---|---|---|---|---|---|---|
| | IOP, | M3 IOP, | lowering | lowering, | preoperative | postoperative |
| Study | mm Hg | mm Hg | (%) | mm Hg | medications | medications |
| Reitsamer et | 21.4 (3.6)* | 15.7^{†} | −25.0^{†} | N.A. | 2.7 (0.9) | 0.5 (0.9) |
| al. | | | | | | |
| Marcos- | 19.1 (5.4)* | N.A. | N.A. | −6.1 (−9.9 to | 2.5 (0.8) | N.A |
| Parra et al. | | | | −0.1)** | | |
| Fea et al. | 23.9 (7.6)* | 15.1^{†} | N.A. | N.A. | 3.0 (1.0) | 0.4^{†} |
| Grover et al. | 25.1 (3.7)* | 16.6 | −32.7^{†} | −8.5^{†} | 3.5 (1.0) | 0.5^{†} |
| | | (5.5)* | | | | |
| Ibáñez- | 22.8 (20.8 | 16.4 (14.3 | N.A. | N.A. | 3.4 (0.8) | N.A. |
| Muñoz et al. | to 24.7)** | to 18.5)** | | | | |
| Laborda- | 21.0 (5.2)* | 14.5 | N.A. | −6.7 (−8.8 to | 2.8 (2.7 to | N.A. |
| Guirao et al. | | (13.6 to | | −4.6) | 3.0)** | |
| | | 15.4)** | | | | |
| Theilig et al. | 24.5 (6.7)* | 16.8 (6.3) | N.A. | N.A. | 3.0 (1.1)* | 1.1 (1.4)* |
| Hengerer et | 32.2 (9.1)* | 14.6^{†} | N.A. | N.A. | 3.1 (1.0)* | −2.7 (1.2)^{‡} |
| Al. | | | | | | |
| Current | 27.0 (7.8)* | 12.2 | −40.8 | −14.8 (−20.1 | 2.3 (0.9)* | 0.1 (0.4)* |
| Study | | (3.4)* | (23.5)* | to −9.5)** | | |

*Mean (Standard deviation)
**Mean (95% confidence interval)
^{†}Data about standard deviation was not provided.
^{‡}Mean reduction from baseline.

TABLE 4
A Comparison of the Clinical Outcomes Between the Current Study and the
Available Evidence.

| | | | | | | | | Needling |
|---|---|---|---|---|---|---|---|---|
| | | | | | | | | Rates at |
| | | | | M24 | | | Mean | Last |
| | Preop | M12 | | IOP, | M24 IOP | Mean | NOHM, | Follow- |
| | IOP, | IOP, | M12 IOP | mm | Lowering, | Preoperative | Last | Up Visit, |
| Study | Hg | mm Hg | Lowering | Hg | mm Hg | NOHM | Visit | n (%) |
| Reitsamer et al. | 21.4 (3.6) * | 14.9 ± 4.5 * | −6.5 ± 5.3 * | 15.2 ± 4.2 * | −6.2 ± 4.9 * | 2.7 ± 0.9 * | 1.1 ± 1.2 * | 83 (41.1) |
| Marcos-Parra et al. | 19.1 | N.A. | −6.7 | N.A. | N.A. | 2.5 (0.8) | 0.2 ± 0.6 * | 13 (20.0) |
| | (5.4) * | | (±12.9 to | | | | | |
| | | | ±0.5) ** | | | | | |
| Fea et al. | 23.9 | 15.5 ± | −7.4 ± 7.9 | N.A. | N.A. | 3.0 (1.0) | 0.5 ± 1.0 * | 79 (46.2) |
| | (7.6) * | 3.9 * | | | | | | |
| Laborda-Guirao et al. | 21.0 | 14.7 | − 6.3 (±8.8 | N.A. | N.A. | 2.8 (2.7 to | 1.1 (0.8 | 7 (8.8) |
| | (5.2) * | (13.9 to | to ±4.4) ** | | | 3.0) ** | to 1.3) ** | |
| | | 15.4) ** | | | | | | |
| Gabbay et al. | 22.1 ± | 15.4 ± | −6.7 ± 6.2 * | 14.5 ± | − 7.6 ± 5.2 | 2.77 ± 1.1 * | 0.5 ± 1.0 * | 57 (37.7) |
| | 6.5 * | 5.9 * | | 3.3 * | | | | |
| Mansouri et al. | 20.0 ± | N.A. | N.A. | 14.1 ± | −6.4 ± 5.9 * | 2.0 ± 1.3 * | 0.6 ± 0.9 * | 58 (45) |
| | 7.5 * | | | 3.7 * | | | | |
| Grover et al. | 25.1 | 15.9 ± | −9.1 | N.A. | N.A. | 3.5 (1.0) | 1.7 | 21 (32.3) |
| | (3.7) * | 5.2 * | (±10.7 to | | | | | |
| | | | 7.5) ** | | | | | |
| Ibáñez-Muñoz et al.¹ | 22.3 | 15.3 | −7.3 (±9.7 | N.A. | N.A. | 3.0 ± 1.0 | 1.2 ± 1.2 | 19 (26.0) |
| | (21.0- | (14.3- | to ±5.0) ** | | | | | |
| | 23.5) ** | 16.3) ** | | | | | | |
| Theilig et al. | 24.5 ± | 16.6 ± | N.A. | N.A. | N.A. | 3.0 ± 1.1 * | 1.4 ± 1.5 * | 42 (42.0) |
| | 6.7 * | 4.8 * | | | | | | |
| Hengerer et al. | 32.2 | 14.2 ± | 32.2 | N.A. | N.A. | 3.1 ± 1.0 * | 0.3 ± 0.7 * | 67 (27.7) |
| | (9.1) * | 4.0 | | | | | | |
| Wanichwecharungruang | 21.6 ± | 15 | 30.6 | 14.6 ± | 32.4 | 2.1 ± 1.4 | 0.5 ± 0.7 | 10 (17.5) |
| and Ratprasatporn | 4.0 | | | 3.5 * | | | | |
| Suba, si et al. | 20.4 ± | 15.0 ± | −6.2 ± 0.9 * | 14.8 ± | −6.4 ± 1.2 * | 3.1 ± 1.0 * | 0.9 ± 1.1 * | 13 (43.3) |
| | 4.8 * | 1.9 * | | 1.9 * | | | | |
| Rauchegger et al. | 23.4 ± | 14.6 ± | 31(20−42) | 14.8 ± | 29(30−41) | 2.7 ± 1.1 * | 1.0 ± 1.2 * | 37 (62) |
| | 7.9 * | 3.6 * | | 4.4 | | | | |
| Current study | 27.0 ± | 14.2 ± | −12.8 | 14.1 ± | −12.9 | 2.3 ± 0.9 * | 1.0 ± 1.4 * | 4 (17.4%) |
| | 7.8 * | 4.6 *± | (±16.7 to | 3.4 *² | (16.9 to | | | |
| | | | ±8.7) ** | | ±8.9) **² | | | |

* Mean (standard deviation).
** Mean (95% confidence interval).
¹Mean IOP lowering was −7.3 (±9.7 to ±5.0) mm Hg in the primary open-angle glaucoma patients and −6.6 (±8.4 to ±4.8) mm Hg in the secondary open-angle glaucoma eyes.
²Month 18 IOP.
† Data about standard deviation were not provided.

What also points to impressive efficacy of the XEN63 system is that with the exception of the Hengerer et al. study (see Hengerer F H. Kohnen T, Mueller M, Conrad-Hengerer I. Ab Interno Gel Implant for the Treatment of Glaucoma Patients With or Without Prior Glaucoma Surgery: 1-Year Results. J Glaucoma. 2017; 26(12):1130-1136. doi: 10.1097/IJG.0000000000000803, the entirety of which is incorporated herein by reference), the baseline IOP of the XEN63 study was higher than that reported by other authors (see Reitsamer H, Sng C. Vera V, Lenzhofer M, Barton K. Stalmans I; Apex Study Group. Two-year results of a multicenter study of the ab interno gelatin implant in medically uncontrolled primary open-angle glaucoma. Graefes Arch Clin Exp Ophthalmol. 2019; 257(5):983-996. doi: 10.1007/s00417-019-04251-z; Marcos Parra M T, Salinas López JA, López Grau N S, Ceausescu A M, Perez Santonja J J. XEN implant device versus trabeculectomy, either alone or in combination with phacoemulsification, in open-angle glaucoma patients. Graefes Arch Clin Exp Ophthalmol. 2019; 257(8):1741-1750. doi: 10.1007/s00417-019-04341-y; Fea A M, Bron A M, Economou M A, Laffi G. Martini E, Figus M, et al. European study of the efficacy of a cross-linked gel stent for the treatment of glaucoma. J Cataract Refract Surg. 2020; 46(3):441-450. doi: 10.1097/j.jcrs.0000000000000065; Grover D S, Flynn W J, Bashford K P, Lewis R A, Duh Y J, Nangia R S, et al. Performance and Safety of a New Ab Intemo Gelatin Stent in Refractory Glaucoma at 12 Months. Am J Ophthalmol. 2017:183:25-36. doi: 10.1016/j.ajo.2017.07.023; Ibáñez-Muñoz A, Soto-Biforcos V S, Chacón-González M, Rúa-Galisteo O, Arrieta-Los Santos A, Lizuain-Abadia M E, et al. One-year follow-up of the XEN® implant with mitomycin-C in pseudoexfoliative glaucoma patients. Eur J Ophthalmol. 2019; 29(3):309-314. doi: 10.1177/1120672118795063; Laborda-Guirao T. Cubero-Parra J M, Hidalgo-Torres A. Efficacy and safety of XEN 45 gel stent alone or in combination with phacoemulsification in advanced open angle glaucoma patients: 1-year retrospective study. Int J Ophthalmol. 2020; 13(8):1250-1256. doi: 10.18240/ijo.2020.08.11; and Theilig T, Rehak M, Busch C, Bormann C, Schargus M. Unterlauft J D. Comparing the efficacy of trabeculectomy and XEN gel microstent implantation for the treatment of primary open-angle glaucoma: a retrospective monocentric comparative cohort study. Sci Rep. 2020:10(1):19337. doi: 10.1038/s41598-020-76551-y; the entireties of each of which are incorporated herein by reference).

Despite its short-term follow-up, this study points to the fact that the XEN63 system, is not only able to achieve lower IOP compared to the XEN45 shunt, but also that a greater lumen size of the device may benefit patients with a higher baseline pressure. The relevance of this finding critically depends upon whether early postoperative pressures may be predictive of long-term success. However, the authors of the XEN63 study have evidence suggesting that lower IOP early postoperative was associated with successful outcomes in patients undergoing trabeculectomy (see e.g., Okimoto S. Kiuchi Y. Akita T, Tanaka J. Using the early postoperative intraocular pressure to predict pressure control after a trabeculectomy. J Glaucoma. 2014; 23(6):410-4. doi: 10.1097/IJG.0b013e318285fd7d; Esfandiari H, Pakravan M, Loewen N A, Yaseri M. Predictive value of early postoperative IOP and bleb morphology in Mitomycin-C augmented trabeculectomy. F1000Res. 2017; 6:1898. doi: 10.12688/f1000research.12904.2, the entireties of each of which are incorporated herein by reference). Moreover, these findings seem to be applicable to the XEN45 shunt (see Fea A M, Bron A M, Economou M A, Laffi G, Martini E, Figus M. et al. European study of the efficacy of a cross-linked gel stent for the treatment of glaucoma. J Cataract Refract Surg. 2020; 46(3):441-450. doi: 10.1097/j.jcrs.0000000000000065; and Karimi A. Lindfield D. Is a Day 1 postoperative review following ab interno Xen gel stent surgery for glaucoma needed?Clin Ophthalmol. 2018; 12:2331-2335. doi: 10.2147/OPTH.S181530; the entireties of each of which are incorporated herein by reference). In a previous study conducted by Applicant (see Fea A M, Bron A M, Economou M A, Laffi G. Martini E. Figus M. et al. European study of the efficacy of a cross-linked gel stent for the treatment of glaucoma. J Cataract Refract Surg. 2020:46(3):441-450. doi: 10.1097/j.jcrs.0000000000000065, the entirety of which is incorporated herein by reference), week-1 and month-1 postoperative IOP significantly correlated with the final IOP and those eyes with a lower IOP at week1 had a higher success rate.

The study also notes that there is limited scientific evidence evaluating the efficacy and safety of a former XEN63 shunt (a shunt having a 63 μm inner diameter, which size of a XEN shunt has never been commercially available). Although in general terms, the results of prior studies of the former XEN63 shunt itself have shown a good efficacy and safety profile, the authors of the present XEN63 system study concluded that due to differences in the surgical technique used to implant the XEN63 shunt (e.g., due to the use of a 27 Gauge needle, and possibly due to the use of MMC, in the presently disclosed XEN63 system), it is difficult to directly compare the present XEN63 study with previous studies related to the former XEN63 shunt. However, there were more favorable trends observed for the XEN63 system.

Regarding safety, the most commonly reported adverse event was choroidal detachment (four eyes), which was successfully resolved without treatment, at month-1 visit. In this study, four eyes (17.4%) had an IOP ≤6 mm Hg at postoperative day one, but they were resolved without consequences. About this subject, Lenzhofer et al. (see Lenzhofer M, Kersten-Gomez I, Sheybani A, Gulamhusein H, Strohmaier C, Hohensinn M, et al. Four-year results of a minimally invasive transscleral glaucoma gel stent implantation in a prospective multi-centre study. Clin Exp Ophthalmol. 2019; 47(5):581-587. doi: 10.1111/ceo.13463, the entirety of which is incorporated herein by reference), using the former XEN63 shunt, reported that three eyes (4.7%) required some intervention (between surgery and end of the study) due to low IOP. Additionally, Lavin-Dapena et al. (Lavin-Dapena C, Cordero-Ros R, D'Anna O, Mogollon I. XEN 63 gel stent device in ship glaucoma surgery: A 5-years follow-up prospective study. Eur J Ophthalmol. 2020:1120672120952033. doi: 10.1177/1120672120952033, the entirety of which is incorporated herein by reference), also evaluating the former XEN63 shunt, found hypotony (similar criterion to those of the XEN63 study) in three eyes (27.3%) at day 1. However, it should be noteworthy that both studies did not use MMC, which in theory might reduce the incidence of hypotony.

Importantly, the XEN63 study also addresses the question of whether a better IOP-lowering effect using the XEN63 system would be associated with a greater risk of hypotony. Despite the larger inner diameter of XEN63 shunt, the incidence of hypotony was surprisingly not significantly different than that observed with XEN45 shunt. Although unclear, the authors of the XEN63 study believe that this may be because the resistance is determined by the subconjunctival bleb (see Fernandez-Garcia A, Zhou Y, Garcia-Alonso M, Andrango H D, Poyales F. Garzón N. Comparing Medium-Term Clinical Outcomes following XEN® 45 and XEN® 63 Device Implantation. J Ophthalmol. 2020; 2020:4796548. doi: 10.1155/2020/4796548, the entirety of which is incorporated herein by reference). However, another explanation for the insignificant difference in the risk of hypotony between the XEN63 shunt and the XEN45 shunt may be related to the surgical technique employed in the XEN63 system. Indeed, because the inserter of the XEN63 system uses a smaller needle (i.e., a 27 Gauge needle) to make the track or incision through the sclera relative to the prior XEN63 inserter device, which utilized a larger gauge needle compared to the XEN63 system, the risk of peritubular filtration should not be greater with the new XEN63 system than with XEN45 shunt because they are both utilizing a 27 Gauge needle that has the same exterior diameter, but for the XEN63 system specifically, the inner lumen has been enlarged to accommodate the larger XEN63 exterior dimensions.

Avoidance of hypotony in the early postoperative phase following glaucoma drainage device surgery is paramount, if serious complications are to be avoided. Hypotony in early postoperative is a common and significant complication that has been associated with delayed visual recovery following trabeculectomy (see Popovic V. Early hypotony after trabeculectomy. Acta Ophthalmol Scand. 1995; 73(3):255-60. doi: 10.1111/j.1600-0420.1995.tb00280.x; and Saeedi O J, Jefferys J L. Solus J F, Jampel H D, Quigley H A. Risk factors for adverse consequences of low intraocular pressure after trabeculectomy. J Glaucoma. 2014; 23(1):e60-8. doi: 10.1097/IJG.0000000000000008, the entireties of each of which are incorporated herein by reference). The reason why early postoperative complications are lower than in trabeculectomy, may be due to the fact that the predetermined lumen allows for a much more controlled outflow as compared to the traditional filtration surgery. Although restriction of outflow using different suturing techniques can improve the safety profile and reduce the rate of early complications observed with trabeculectomy, this carries the disadvantage of manipulation of the sutures in the postoperative period (see Lin S. Building a safer trabeculectomy. Br J Ophthalmol. 2006; 90(1):4-5. doi: 10.1136/bjo.2005.078568, the entirety of which is incorporated herein by reference).

In this XEN63 study, early hypotony was not related with ocular complications or visual acuity loss. In the overall study sample, mean visual acuity did not change over the course of the study. Although at day-7, four eyes (17.4%) had a ≥2-line worsening in BCVA as compared to baseline, two eyes recovered within a month. Moreover, it should be highlighted that at month 3, eight eyes (34.8%) showed a ≥2-line improvement in BCVA as compared to baseline.

Also impressively, the present XEN63 study shows that with regard to needling, three eyes (13.0%) underwent postoperative needling—with two of them being preventative measures and only one of them due to increased IOP. Nevertheless, even a total of three represents a low needling rate as compared with that reported in previous XEN45 shunt studies (see De Gregorio A. Pedrotti E. Russo L, Morselli S. Minimally invasive combined glaucoma and cataract surgery: clinical results of the smallest ab interno gel stent. Int Ophthalmol. 2018; 38(3):1129-1134. doi: 10.1007/s10792-017-0571-x; Reitsamer H, Sng C, Vera V. Lenzhofer M, Barton K, Stalmans I; Apex Study Group. Two-year results of a multicenter study of the ab interno gelatin implant in medically uncontrolled primary open-angle glaucoma. Graefes Arch Clin Exp Ophthalmol. 2019; 257(5):983-996. doi: 10.1007/s00417-019-04251-z; Chatzara A, Chronopoulou I, Theodossiadis G, Theodossiadis P, Chatziralli I. XEN Implant for Glaucoma Treatment: A Review of the Literature. Semin Ophthalmol. 2019; 34(2):93-97. doi: 10.1080/08820538.2019.1581820; Marcos Parra M T, Salinas Lopez J A, López Grau N S, Ceausescu A M, Pérez Santonja J J. XEN implant device versus trabeculectomy, either alone or in combination with phacoemulsification, in open-angle glaucoma patients. Graefes Arch Clin Exp Ophthalmol. 2019; 257(8):1741-1750. doi: 10.1007/s00417-019-04341-y; Fea A M, Bron A M, Economou M A, Laffi G, Martini E, Figus M, et al. European study of the efficacy of a cross-linked gel stent for the treatment of glaucoma. J Cataract Refract Surg. 2020; 46(3):441-450. doi: 10.1097/j.jcrs.0000000000000065; and Fea A M, Durr G M, Marolo P, Malinverni L, Economou M A, Ahmed I). XEN<sup>®</sup> Gel Stent: A Comprehensive Review on Its Use as a Treatment Option for Refractory Glaucoma. Clin Ophthalmol. 2020:14:1805-1832. doi: 10.2147/OPTH.S178348, the entirety of which is incorporated herein by reference).

Applicant also notes that although the XEN63 study has certain limitations, there are similar countervailing considerations that justify the strong performance of the XEN63 system. For example, although the XEN63 study has a retrospective design and potential bias and confounding factors inherent of retrospective studies, strict inclusion/exclusion criteria were selected to minimize their impact. Also, although the XEN63 study had limited follow-up time, the assessment of short-term clinical outcomes is useful at least because early postoperative TOP seems to be associated with the success of the procedure. Additionally, although there was not a sample size calculation at the start of the study, according to the results of the study, the power for detecting mean TOP lowering and ocular hypotensive drug reduction, between baseline and month 3, was 99% each, respectively. Finally, although there was no simultaneous control group using the XEN45 shunt (such as a head-to-head randomized clinical trial), the XEN63 study relies on numerous prior XEN45 shunt studies to provide comparative data.

Indeed, despite these limitations, the results of the current study clearly demonstrate the surprising effectiveness of embodiments of the XEN63 system disclosed herein in providing an effective and safe surgical procedure in OAG patients. As discussed herein, such embodiments of the XEN63 system significantly lowered IOP and reduced the number of antiglaucoma medications, with a good safety profile. Further, such embodiments of the XEN63 system's limited incidence of hypotony, in combination with the optional use of MMC (both in terms of concentration and area of injection), may allow to treat more advanced patients and to obtain a lower target-IOP in the long-term. These and other factors demonstrate the surprisingly effective, unexpected results of embodiments of the XEN63 system, especially considering the fact that such embodiments are contrary to medical experts' traditional practice, experience, and teachings.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. Unless otherwise expressed, reference to an element in the singular is not intended to mean “one and only one” unless explicitly stated, but rather is meant to mean “one or more.” In addition, it is not necessary for a device or method to address every problem that is solvable by different embodiments of the disclosure in order to be encompassed within the scope of the disclosure.

### Illustration of Subject Technology as Clauses

Various examples of aspects of the disclosure are described below as clauses for convenience. These are provided as examples, and do not limit the subject technology.

Clause 1. A system for treating glaucoma, comprising: an intraocular shunt made of a cross-linked gelatin, the intraocular shunt having a shunt outer diameter of between about 170 μm to about 260 μm, and the intraocular shunt defining at least one interior flow path having a shunt inner diameter of between about 50 μm and about 70 μm; and a needle having a lumen to carry the intraocular shunt, the lumen having a lumen inner diameter of between about 220 to about 280 μm.

Clause 2. The system of Clause 1, wherein an annular gap between an outer surface of the shunt and an inner surface of the lumen is between about 0 μm to about 20 μm.

Clause 3. The system of any of the preceding Clauses, wherein the shunt outer diameter is expandable.

Clause 4. The system of Clause 3, wherein the shunt outer diameter expands to a maximum shunt outer diameter of about 260 μm.

Clause 5. The system of any of the preceding Clauses, wherein the shunt inner diameter is about 63 μm.

Clause 6. The system of any of the preceding Clauses, wherein the intraocular shunt has a shunt wall thickness of between about 88 μm to about 99 μm.

Clause 7. The system of any of the preceding Clauses, wherein the intraocular shunt has a shunt length of about 6 millimeters.

Clause 8. The system of any of the preceding Clauses, wherein the lumen inner diameter is about 260 μm.

Clause 9. The system of any of the preceding Clauses, wherein the needle has a needle outer diameter of between about 400 μm to about 420 μm.

Clause 10. The system of any of the preceding Clauses, wherein the needle has a needle wall thickness of between about 70 μm to about 80 μm.

Clause 11. The system of any of the preceding Clauses, further comprising a movable plunger disposed within the lumen, wherein the plunger is configured to advance the shunt through the lumen.

Clause 12. The system of Clause 11, further comprising a deployment mechanism configured to selectively move the plunger to advance the shunt.

Clause 13. A method to treat glaucoma, the method comprising: advancing a needle through the sclera of the patient to create an opening, wherein the needle has a needle diameter of between about 400 μm to about 420 μm; advancing an intraocular shunt through a lumen of the needle and into the opening, wherein the intraocular shunt has a shunt outer diameter of between about 240 to 260 μm and the lumen has a lumen inner diameter of between about 220 μm to about 280 μm for reducing any gap between the shunt and the opening to minimize peritubular flow therebetween; and permitting flow through the intraocular shunt, wherein the shunt has a shunt inner diameter of between about 50 μm to about 70 μm.

Clause 14. The method of Clause 13, further comprising permitting expansion of the intraocular shunt.

Clause 15. The method of Clause 14, wherein the shunt outer diameter is limited to approximately the lumen inner diameter during expansion.

Clause 16. The method of any of Clauses 13-15, further comprising expanding the shunt outer diameter to approximately an inner diameter of the opening when the shunt is outside of the lumen of the needle.

Clause 17. The method of any of Clauses 13-16, further comprising cutting the sclera via a tip of the needle to form the opening.

Clause 18. A system for treating glaucoma, comprising: an intraocular shunt made of a cross-linked gelatin, the intraocular shunt having a shunt outer diameter of between about 170 μm to about 260 μm, and the intraocular shunt defining at least one interior flow path having a shunt inner diameter of about 63 μm, and a needle having a lumen to carry the intraocular shunt, the needle having a needle outer diameter of between about 400 μm to about 420 μm and the lumen having a lumen inner diameter of about 260 μm.

Clause 19. The system of Clause 18, wherein the intraocular shunt has a shunt wall thickness of between about 88 μm to about 99 μm.

Clause 20. The system of any of Clauses 18 to 19, wherein the needle has a needle wall thickness of between about 70 μm to about 80 μm.

### FURTHER CONSIDERATIONS

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

A reference to an element in the singular is not intended to mean one and only one unless specifically so stated, but rather one or more. For example, “a” module may refer to one or more modules. An element proceeded by “a,” “an,” “the,” or “said” does not, without further constraints, preclude the existence of additional same elements.

Headings and subheadings, if any, are used for convenience only and do not limit the invention. The word exemplary is used to mean serving as an example or illustration. To the extent that the term includes, have, or the like is used, such term is intended to be inclusive in a manner similar to the term comprise as comprise is interpreted when employed as a transitional word in a claim. Relational terms such as first and second and the like may be used to distinguish one entity or action from another without necessarily requiring or implying any actual such relationship or order between such entities or actions.

Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A phrase “at least one of” preceding a series of items, with the terms “and” or “or” to separate any of the items, modifies the list as a whole, rather than each member of the list. The phrase “at least one of” does not require selection of at least one item; rather, the phrase allows a meaning that includes at least one of any one of the items, and/or at least one of any combination of the items, and/or at least one of each of the items. By way of example, each of the phrases “at least one of A, B, and C” or “at least one of A, B, or C” refers to only A, only B, or only C; any combination of A, B, and C; and/or at least one of each of A, B, and C.

It is understood that the specific order or hierarchy of steps, operations, or processes disclosed is an illustration of exemplary approaches. Unless explicitly stated otherwise, it is understood that the specific order or hierarchy of steps, operations, or processes may be performed in different order. Some of the steps, operations, or processes may be performed simultaneously. The accompanying method claims, if any, present elements of the various steps, operations or processes in a sample order, and are not meant to be limited to the specific order or hierarchy presented. These may be performed in serial, linearly, in parallel or in different order. It should be understood that the described instructions, operations, and systems can generally be integrated together in a single software/hardware product or packaged into multiple software/hardware products.

In one aspect, a term “coupled” or the like may refer to being directly coupled. In another aspect, a term “coupled” or the like may refer to being indirectly coupled.

Terms such as top, bottom, front, rear, side, horizontal, vertical, and the like refer to an arbitrary frame of reference, rather than to the ordinary gravitational frame of reference. Thus, such a term may extend upwardly, downwardly, diagonally, or horizontally in a gravitational frame of reference.

The disclosure is provided to enable any person skilled in the art to practice the various aspects described herein. In some instances, well-known structures and components are shown in block diagram form in order to avoid obscuring the concepts of the subject technology. The disclosure provides various examples of the subject technology, and the subject technology is not limited to these examples. Various modifications to these aspects will be readily apparent to those skilled in the art, and the principles described herein may be applied to other aspects.

All structural and functional equivalents to the elements of the various aspects described throughout the disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

What is claimed is:

1. A system for treating glaucoma, comprising:
an intraocular shunt made of a cross-linked gelatin, the intraocular shunt having a shunt outer diameter of between about 170 μm to about 260 μm, and the intraocular shunt defining at least one interior flow path having a shunt inner diameter of between about 50 μm and about 70 μm; and
a needle having a lumen to carry the intraocular shunt, the lumen having a lumen inner diameter of between about 220 to about 280 μm.

2. The system of claim 1, wherein an annular gap between an outer surface of the shunt and an inner surface of the lumen is between about 0 μm to about 20 μm.

3. The system of claim 1, wherein the shunt outer diameter is expandable.

4. The system of claim 3, wherein the shunt outer diameter expands to a maximum shunt outer diameter of about 260 μm.

5. The system of claim 1, wherein the shunt inner diameter is about 63 μm.

6. The system of claim 1, wherein the intraocular shunt has a shunt wall thickness of between about 88 μm to about 99 μm.

7. The system of claim 1, wherein the intraocular shunt has a shunt length of about 6 millimeters.

8. The system of claim 1, wherein the lumen inner diameter is about 260 μm.

9. The system of claim 1, wherein the needle has a needle outer diameter of between about 400 μm to about 420 μm.

10. The system of claim 1, wherein the needle has a needle wall thickness of between about 70 μm to about 80 μm.

11. The system of claim 1, further comprising a movable plunger disposed within the lumen, wherein the plunger is configured to advance the shunt through the lumen.

12. The system of claim 11, further comprising a deployment mechanism configured to selectively move the plunger to advance the shunt.

13. A method to treat glaucoma, the method comprising:
advancing a needle through the sclera of the patient to create an opening, wherein the needle has a needle diameter of between about 400 μm to about 420 μm;
advancing an intraocular shunt through a lumen of the needle and into the opening, wherein the intraocular shunt has a shunt outer diameter of between about 240 to 260 μm and the lumen has a lumen inner diameter of between about 220 μm to about 280 μm for reducing any gap between the shunt and the opening to minimize peritubular flow therebetween, and
permitting flow through the intraocular shunt, wherein the shunt has a shunt inner diameter of between about 50 μm to about 70 μm.

14. The method of claim 13, further comprising permitting expansion of the intraocular shunt.

15. The method of claim 14, wherein the shunt outer diameter is limited to approximately the lumen inner diameter during expansion.

16. The method of claim 13, further comprising expanding the shunt outer diameter to approximately an inner diameter of the opening when the shunt is outside of the lumen of the needle.

17. The method of claim 13, further comprising cutting the sclera via a tip of the needle to form the opening.

18. A system for treating glaucoma, comprising:
an intraocular shunt made of a cross-linked gelatin, the intraocular shunt having a shunt outer diameter of between about 170 μm to about 260 μm, and the intraocular shunt defining at least one interior flow path having a shunt inner diameter of about 63 μm; and
a needle having a lumen to carry the intraocular shunt, the needle having a needle outer diameter of between about 400 μm to about 420 μm and the lumen having a lumen inner diameter of about 260 μm.

19. The system of claim 18, wherein the intraocular shunt has a shunt wall thickness of between about 88 μm to about 99 μm.

20. The system of claim 18, wherein the needle has a needle wall thickness of between about 70 μm to about 80 μm.
